(19) United States
(12) Patent Application Publication
(10) Pub. No.: US 2020/0290980 A1
(43) Pub. Date: Sep. 17, 2020

(54) POLYCATENAR LIGANDS AND HYBRID NANOPARTICLES MADE THEREFROM

(71) Applicants: RHODIA OPERATIONS, Paris (FR); THE TRUSTEES OF THE UNIVERSITY OF PENNSYLVANIA, Philadelphia, PA (US)

(72) Inventors: Bertrand DONNIO, Strasbourg (FR); Davit JISHKARIANI, Philadelphia, PA (US); Benjamin DIROLL, Philadelphia, PA (US); Christopher MURRAY, Bala Cynwyd Lower Merion, PA (US); Lawrence Alan HOUGH, Yongsan-gu, Seoul (KR); Matteo CARGNELLO, Palo Alto, CA (US); Stan NAJMR, Philadelphia, PA (US); Katherine C. ELBERT, Ringoes, NJ (US)

(21) Appl. No.: 16/086,095

(22) PCT Filed: Mar. 17, 2017
(86) PCT No.: PCT/US2017/023003

§ 371 Date: Sep. 18, 2018

Related U.S. Application Data
(60) Provisional application No. 62/310,047, filed on Mar. 18, 2016.
(60) Provisional application No. 62/424,133, filed on Nov. 18, 2016.

(51) Int. Cl. C07D 249/04; C09K 11/02; C09K 11/88

(52) U.S. Cl. CPC C07D 249/04; B82Y 30/00; C09K 11/883; C09K 11/025

(57) ABSTRACT

Described herein are polycatenar ligand compounds and their use in the production of hybrid nanoparticles, typically nanocrystals. The present disclosure also relates to films containing the hybrid nanoparticles described herein and their use.

### CROSS-REFERENCE TO RELATED APPLICATIONS

This application claims the priority of U.S. Provisional Application Nos. 62/310,047, filed Mar. 18, 2016, and 62/424,133, filed Nov. 18, 2016, both of which are hereby incorporated by reference in their entireties.

### FIELD OF THE INVENTION

The present disclosure relates to polycatenar ligand compounds and their use in the production of hybrid nanoparticles, typically nanocrystals. The present disclosure also relates to films containing the hybrid nanoparticles described herein and their use, for example, in applications ranging from solid-state devices, including plasmonic enhancement of optoelectronic devices, bio-related applications such as bio-imaging, theranostic, and drug delivery to polymeric additives for enhanced properties. The ligand shells may be useful for modulating solubility (including amphiphilicity) and surface wetting properties.

### BACKGROUND

Ligand coatings can be used to change the properties of and the growth dynamics of colloidal nanoparticles. Properties capable of alteration by ligand coatings include, but are not limited to, solubility, aggregation/dispersion behavior, self-assembly, thermal stability, optical properties, electronic conduction, catalytic activity, and magnetism. With some important exceptions, the toolset of available ligands is typically limited to commercial sources, imposing constraints on the functionality which can be engineered into the organic shell of an inorganic, typically metallic, nanoparticle and leaving tremendous parameter space for synthetic design of tailored ligands.

For nanoparticles, such as, for example, nanocrystals, in hydrophobic media, ligands are typically long-chain alkyl amines, phosphines, carboxylic acids, phosphonic acids, or thiols. However, many past efforts for producing such hydrophobic nanoparticles rely on post-synthetic ligand-exchange, i.e., formation of the nanoparticle using a first ligand and then exchanging the first ligand with a second ligand at a later step after formation. While some instances of direct synthesis are known, such instances are limited to Au nanocrystals made near room temperature. Moreover, in these examples of direct synthesis, size distribution is relatively large (19% or higher) or unmentioned and the scale of self-assembly is limited (≤60 nm, or about 10 particles). Direct synthesis of nanoparticles into designed structures is of crucial importance for the engineering of new materials with tunable functions and for the subsequent bottom-up fabrication of functional devices.

Also of importance are nanoparticles, for example, nanocrystals, containing rare earth elements. Rare earth nanoparticles are a valuable class of nanomaterial due to their ability to bring the attractive bulk properties of the rare earth elements, such as their magnetic, catalytic, and optical properties, to processing and biomedical applications. Currently, solvothermal synthesis offers good control of the morphology and monodispersity of rare earth nanoparticles, such as nanocrystals. However, the morphology and monodispersity of such rare earth nanoparticles remain significantly dependent on a number of reaction parameters, such as temperature, time, and ligand environment, among others, leading to development of synthetic methods mainly by trial-and-error.

Thus, there is an ongoing, unresolved need for a platform suitable for achieving tight control over the formation of nanoparticles, particularly nanocrystals, while still preserving ordered assemblies of such nanoparticles over large areas with high uniformity. Herein, new polycatenar ligand compounds and their use in the production of hybrid nanoparticles, typically nanocrystals, are described.

The use of new polycatenar ligand compounds in the synthesis of rare earth nanoparticles, typically nanocrystals, is also described herein.

### SUMMARY OF THE INVENTION

An objective of the present invention is providing new polycatenar ligands with diverse anchoring functionality for forming hybrid nanoparticles, particularly nanocrystals, capable of self-assembling into ordered assemblies over large areas with high uniformity.

Another objective of the present invention is to provide a strategy that allows great synthetic flexibility and access to a variety of new polycatenar ligands that are suitable for the direct synthesis of the hybrid nanoparticles described herein.

Therefore, in a first aspect, the present disclosure relates to a compound represented by formula (I)

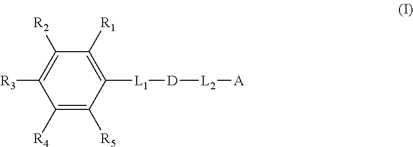

wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
D is a divalent moiety selected from the group consisting of

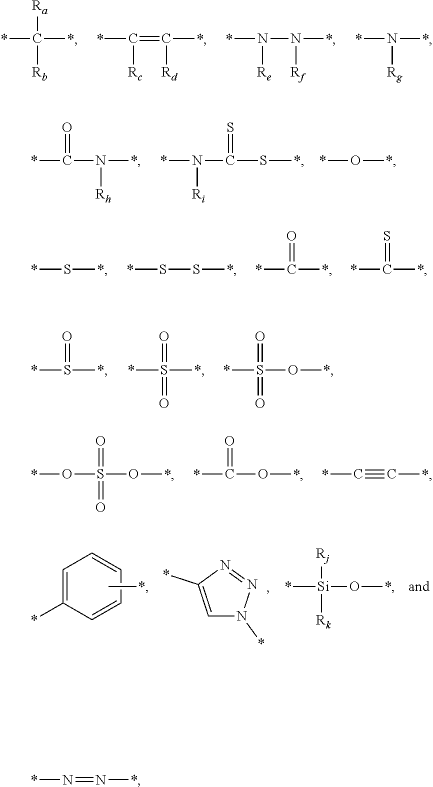

wherein each occurrence of Rₐ-Rₖ are each, independently, H, halogen, or hydrocarbyl; and
A is —COOR₇, —NR₈R₉, —PO₃R₁₀R₁₁, —CN, —SR₁₂, —(SR₁₃)CH₂(SR₁₄), —Si(OR₁₅)₃, —H, or —OR₁₆, wherein each occurrence of R₇-R₁₆ are each, independently, H or hydrocarbyl.

In a second aspect, the present disclosure relates to a method for producing the compound represented by formula (I), the method comprising:
reacting a compound represented by the structure of formula (II):

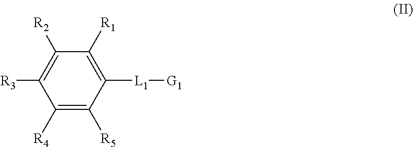

with a compound represented by the structure of formula (III):

G₂-L₂-A (III)
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
A is —COOR₇, —NR₈R₉, —PO₃R₁₀R₁₁, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl; and
each occurrence of G₁ is a reactive group capable of reacting with the reactive group G₂, and
G₂ is a reactive group capable of reacting with the reactive group G₁.

In a third aspect, the present disclosure relates to a hybrid nanoparticle comprising:
(a) a metallic core, and
(b) a compound represented by formula (I) attached to the surface of the metallic core.

In a fourth aspect, the present disclosure relates to a method for producing the hybrid nanoparticle described herein, the method comprising:
forming the metallic core in the presence of a compound represented by formula (I); thereby producing the hybrid nanoparticle.

In a fifth aspect, the present disclosure relates to a film comprising a plurality of the hybrid nanoparticles described herein.

In a sixth aspect, the present disclosure relates to the use of a compound represented by formula (I) for producing a hybrid nanoparticle comprising a metallic core.

In a seventh aspect, the present disclosure relates to a method for making nanoparticles comprising a rare earth element, the method comprising:
(a) heating one or more reaction vessels, each said vessel containing a reaction mixture comprising a rare earth-containing precursor compound, and
(b) recovering the nanoparticles formed in the one or more reaction vessels in step (a).

### BRIEF DESCRIPTION OF THE FIGURES

FIG. 1 shows the thermogravimetric analysis (TGA) of 20b-capped 2.8 nm CdSe NCs compared to oleate-capped 2.8 nm CdSe to under air flow.

FIG. 2 shows (a) the proton NMR spectrum of ligand 20d and (b) NMR spectrum of 20d-capped 2.4 nm CdSe NCs showing the matching signals.

FIG. 3 shows (a) the infrared absorption spectrum of 20d-capped CdSe NCs and (b) oleate-capped CdSe NCs.

FIG. 4 shows (a) ¹H NMR spectra of polycatenar ligand 20a with corresponding signal assignments and (b) 20a-capped ZnO nanocrystals.

FIG. 5 shows (a) the visible and near-IR absorption spectra and (b) the X-ray diffraction patterns for typical examples of the hybrid nanoparticles described herein.

FIG. 6 shows (a) the optical absorption data, (b) SAXS data, and (c) SANS data for 20a-capped CdSe (bottom curve) and oleate-capped CdSe (top curve) NCs.

FIG. 7 shows the small-angle x-ray scattering from 20a- and 20d-capped CdSe NCs prepared by drop-casting on to a quartz coverslip.

FIG. 8 shows the optical absorption spectra of several CdSe NC syntheses using the inventive compounds 20a-20j and 20l described herein.

FIG. 9 shows the TEM Micrographs of colloidal nanocrystals synthesized the inventive ligands described herein, labeled by the inorganic material.

FIG. 10 shows 20b-capped 2.8 nm PbS NCs self-assembled on DEG into a bcc structure.

FIG. 11 shows 20b-capped 3.5 nm PbSe NCs self-assembled on DEG into a bcc structure.

FIG. 12 shows 20i-capped 7.0 nm ZnO NCs drop-cast on to a TEM grid.

FIG. 13 shows 20n-capped 2.5 nm Au NCs drop-cast on a TEM grid.

FIG. 14 shows 20d-capped 2.4 nm CdSe NCs self-assembled into a bcc superlattice.

FIG. 15 shows 20b-capped 2.6 nm CdSe NCs self-assembled into a bcc superlattice.

FIG. 16 shows 20a-capped 2.8 nm CdSe NCs self-assembled into a bcc superlattice.

FIG. 17 shows the characteristic twin boundary of bcc superlattice composed of 20b-capped CdS NCs.

FIG. 18 shows 20c-capped CdSe forming short-range hcp (hexagonal close-packed) lattices after drop-casting.

FIG. 19 shows the hcp domain of 20f-capped CdSe NCs.

FIG. 20 shows the product from the synthesis of CdSe NCs with ligand 20e.

FIG. 21 shows 20h-capped 2.5 nm CdSe NCs drop-cast from a dilute dispersion.

FIG. 22 shows 20j-capped 2.8 nm CdSe NCs drop-cast from a dilute dispersion.

FIG. 23 shows the product from the synthesis of CdSe NCs with ligand 20k.

FIG. 24 shows (a) (001) projection of bcc 20b-capped 2.8 nm CdSe, with inset of 20b-capped 2.4 nm CdS, (b) (001) projection of hcp 20i-capped 7.0 nm ZnO, and (c) close-packed hexagonal monolayer of 20b-capped 5.5 nm PbSe NCs; (d) bcc-type self-assembly of 2.8 nm CdSe NCs prepared by direct synthesis with ligand 20a; (e) hcp-type assembly of 2.8 nm CdSe NCs prepared by synthesis with oleic acid ligands; (f, g) 3.5 nm CdSe NCs capped with 20i ligand self-assembled into hcp, fcc, and bcc superlattices; (h) hcp superlattice of 20o-capped 6.5 nm Au NCs. Inset shows an fcc assembly of the same sample; (i) 20o-capped 3.0 nm Au NCs in a bcc superlattice. The inset shows another region of bcc superlattices of the sample including a characteristic twin boundary.

FIG. 25 shows the spontaneous formation of CaCu₅-like BNSL domains in a drop-casted sample of CdSe NCs synthesized with ligand 20l.

FIG. 26 shows MgZn₂-type BNSL self-assembled from oleate-capped 5.3 nm CdSe NCs and 20d-capped 2.4 nm CdSe NCs.

FIG. 27 shows MgZn₂-type BNSLs self-assembled from 20b-capped 3.5 nm PbSe NCs and 20b-capped 2.8 nm CdSe NCs.

FIG. 28 shows a possible AlB₂-type BNSL self-assembled from 20b-capped 5.5 nm PbSe NCs and 20d-capped 2.4 nm CdSe NCs.

FIG. 29 shows an AlB₂-type BNSL self-assembled from 20b-capped 5.5 nm PbSe NCs and 20d-capped 2.4 nm CdSe NCs.

FIG. 30 shows CuAu BNSLs with antiphase boundaries formed from 20o-capped 6.5 nm Au NCs and oleate-capped 2.8 nm CdSe NCs.

FIG. 31 shows a binary liquid crystalline phase formed from 20o-capped 6.5 nm Au NCs and oleate-capped 2.8 nm CdSe NCs.

FIG. 32 shows a binary liquid crystalline phase formed from 20o-capped 6.5 nm Au NCs and oleate-capped 2.8 nm CdSe NCs.

FIG. 33 shows TEM micrographs of (a) NaZn₁₃, (b) Cu₃Au, and (c) CuAu BNSLs composed of different stoichiometries of 20b-capped 5.5 nm PbSe and 20d-capped 2.4 nm CdSe NCs; (d) CaCu₅ BNSL composed of 20i-capped 3.5 nm CdSe and 20i-capped 7.0 nm ZnO NCs; (e) MgZn₂ and (f) CaCu₅ BNSLs composed of different stoichiometries of 20i-capped 3.5 nm CdSe and 20b-capped 2.4 nm CdS NCs. The inset of (f) shows 12-fold symmetric defects which occur in mixtures of the two NCs. (g) Cu₃Au-type, (h) CuAu-type, and (i) AlB₂-type BNSLs formed from different stoichiometries of oleate-capped 2.8 nm CdSe and 20o-capped 6.5 nm Au NCs.

FIG. 34 shows representative TEM images of DyF₃ plates obtained from (A) a traditional solvothermal reaction vessel and (B) from a microreactor, respectively; DyF₃ elongated plates obtained from an equimolar ratio of oleic acid and polycatenar ligand in (C) a microreactor and (D) a traditional solvothermal reaction vessel, respectively.

FIG. 35 shows β-NaYF₄:0.2% Tm, 20% Yb upconverting nanocrystals synthesized from the microreactor vessel at 340° C. for 40 min. (A) Transmission electron microscopy confirms the expected hexagonal prism morphology; (B) Powder x-ray diffraction with β-NaYF₄ peak assignment; (C) Optical response upon 980 nm excitation.

FIG. 36 shows resulting DyF₃ nanoparticles by % molar replacement of oleic acid by the polycatenar ligands. (A) 0% (no polycatenar ligand); (B) 20%; (C) 40%; (D) 50%; (E) 80%; and (F) 100% molar replacement (no oleic acid).

FIG. 37 shows low-magnification image of elongated plates of DyF₃ achieved at a 50% molar replacement of oleic acid with the polycatenar ligands from the microreaction setup.

FIG. 38 shows the X-ray diffraction pattern of the DyF₃ syntheses in the presence of inventive ligands. The peak assignment is indicative of the α-phase DyF₃.

FIG. 39 shows the analysis of a DyF3 elongated plate obtained from 50% molar replacement of oleic acid with inventive polycatenar ligands. (A) High magnification TEM image. (B) A 3D reconstruction of the plate from tilt tomography data. (C) Illustration highlighting the curvature of the plate.

FIG. 40 shows representative TEM images of rare earth (RE) nanocrystals obtained from an equimolar mixture of oleic acid to polycatenar ligand in microreactors, resulting in (A) circular platelets of LaF₃ and (B) EuF₃ and (C) octahedra of LiYF₄ and (D) LiErF₄.

FIG. 41 shows (A) a TEM image of three DyF₃ screw-dislocated nanoparticles; (B), (C) selected high-magnification images of DyF₃ nanoparticles; (D), (E) 3D reconstructions of the particles shown in (B) and (C), respectively, after a tilt-series.

### DETAILED DESCRIPTION

As used herein, the terms “a”, “an”, or “the” means “one or more” or “at least one” unless otherwise stated.

As used herein, the term “comprises” includes “consists essentially of” and “consists of.” The term “comprising” includes “consisting essentially of” and “consisting of.”

The phrase “free of” means that there is no external addition of the material modified by the phrase and that there is no detectable amount of the material that may be observed by analytical techniques known to the ordinarily-skilled artisan, such as, for example, gas or liquid chromatography, spectrophotometry, optical microscopy, and the like.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

As used herein, the terminology “(Cx-Cy)” in reference to an organic group, wherein x and y are each integers, means that the group may contain from x carbon atoms to y carbon atoms per group.

As used herein, the term “hydrocarbyl” means a monovalent radical formed by removing one hydrogen atom from a hydrocarbon, typically a (C₁-C₄₀) hydrocarbon, more typically a (C₁-C₃₀) hydrocarbon. Hydrocarbyl groups may be straight, branched or cyclic, and may be saturated or unsaturated. Examples of hydrocarbyl groups include, but are not limited to, alkyl, fluoroalkyl, alkenyl, alkynyl, cycloalkyl, aryl, and arylalkyl.

As used herein, the term “alkyl” means a monovalent straight or branched saturated hydrocarbon radical, more typically, a monovalent straight or branched saturated (C₁-C₄₀)hydrocarbon radical, such as, for example, methyl, ethyl, n-propyl, isopropyl, n-butyl, isobutyl, tert-butyl, hexyl, 2-ethylhexyl, octyl, dodecyl, hexadecyl, octadecyl, eicosyl, behenyl, tricontyl, tetracontyl, and so on. As used herein, the term “cycloalkyl” means a monovalent saturated cyclic hydrocarbon radical, more typically a saturated cyclic (C₅-C₂₂) hydrocarbon radical, such as, for example, cyclopentyl, cycloheptyl, cyclooctyl.

As used herein, the term “fluoroalkyl” means an alkyl radical as defined herein, more typically a (C₁-C₄₀) alkyl radical that is substituted with one or more fluorine atoms. Thus, a fluoroalkyl group includes alkyl radicals that may be partially or completely substituted by fluorine atoms. Herein, fluoroalkyl groups that are completely substituted by fluorine atoms are referred to as being perfluorinated. Examples of fluoroalkyl groups include, for example, difluoromethyl, trifluoromethyl, perfluoroalkyl, 1H,1H,2H,2H-perfluorooctyl, perfluoroethyl, and heptadecafluorododecyl.

As used herein, the term “aryl” means a monovalent group having at least one aromatic ring. As understood by the ordinarily-skilled artisan, an aromatic ring has a plurality of carbon atoms, arranged in a ring and has a delocalized conjugated π electron system, typically represented by alternating single and double bonds. Aryl radicals include monocyclic aryl and polycyclic aryl. Polycyclic aryl means a monovalent group having two or more aromatic rings wherein adjacent rings may be linked to each other by one or more bonds or divalent bridging groups or may be fused together. Examples of aryl radicals include, but are not limited to, phenyl, anthracenyl, naphthyl, phenanthrenyl, fluorenyl, and pyrenyl.

As used herein, the term “arylalkyl” refers to an alkyl group as defined herein that is substituted by at least one aryl group. Examples of arylalkyl groups include, but are not limited to, phenylmethyl (benzyl), phenylethyl, phenylpropyl, and phenylbutyl.

As used herein, the term “hydrocarbylene” means a divalent radical formed by removing two hydrogen atoms from a hydrocarbon, typically a (C₁-C₄₀) hydrocarbon. Hydrocarbylene groups may be straight, branched or cyclic, and may be saturated or unsaturated. Examples of hydrocarbylene groups include, but are not limited to, alkylene groups, such as methylene, ethylene, propylene, and butylene, among others, and arylene groups, such as 1,2-benzene, 1,3-benzene, 1,4-benzene, and 2,6-naphthalene, among others.

Any substituent or radical described herein may optionally be substituted at one or more carbon atoms with one or more, same or different, substituents described herein. For instance, a hydrocarbyl group may be further substituted with an aryl group or an alkyl group. Any substituent or radical described herein may also optionally be substituted at one or more carbon atoms with one or more substituents selected from the group consisting of halogen, such as, for example, F, Cl, Br, and I; nitro (NO₂), cyano (CN), and hydroxy (OH). When a substituent or radical described herein is substituted at one or more carbon atoms with one or more substituents selected from the group consisting of halogen, such as, for example, F, Cl, Br, and I, the substituent or radical is said to be halogenated.

Any substituent or radical described herein may optionally be substituted with a carboxylate group. Herein, a carboxylate group refers to a —CO₂M group, wherein M may be H⁺ or an alkali metal ion, such as, for example, Na⁺, Li⁺, K⁺, Rb⁺, Cs⁺; or ammonium (NH₄⁺). For example, an aryl group may be substituted with a CO₂H group.

The present disclosure relates to a compound represented by formula (I)

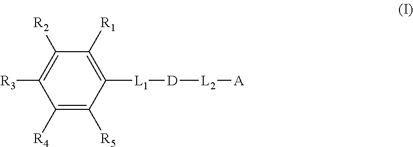

wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
D is a divalent moiety selected from the group consisting of

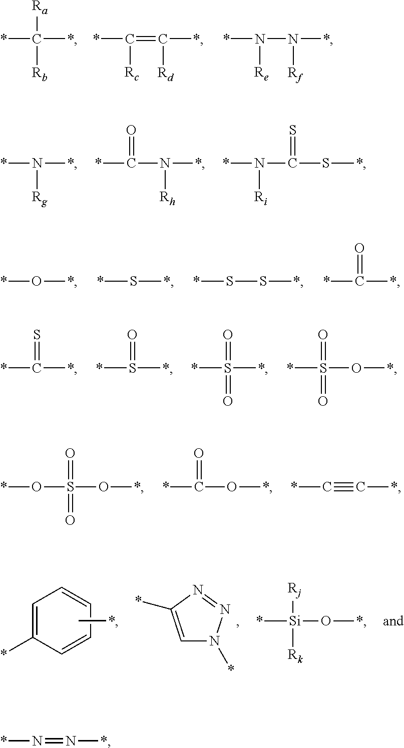

wherein each occurrence of Rₐ-Rₖ are each, independently, H, halogen, or hydrocarbyl; and
A is —COOR₇, —NR₈R₉, —PO₃R₁₀R₁₁, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl.

In an embodiment, R₁, R₂, R₃, R₄, and R₅ are each, independently, H, alkyl, fluoroalkyl, or —OR₆, wherein each occurrence of R₆ is alkyl, arylalkyl, or fluoroalkyl.

In another embodiment, R₁, R₂, R₃, R₄, and R₅ are each, independently, H or —OR₆, wherein each occurrence of R₆ is (C₁-C₂₀)alkyl, (C₁-C₂₀)arylalkyl, or (C₁-C₂₀)fluoroalkyl.

In yet another embodiment, R₁ and R₅ are each H.

In an embodiment, R₂ is H; R₃ and R₄ are each, independently —OR₆.

In an embodiment, R₃ is H; R₂ and R₄ are each, independently —OR₆.

In an embodiment, R₂ and R₄ are each H; and R₃ is —OR₆.

In another embodiment, R₂, R₃, and R₄ are each —OR₆.

In an embodiment, R₆ is (C₁₂-C₁₈)alkyl.

In an embodiment, R₆ is benzyl.

In an embodiment, R₆ is (C₁₂)fluoroalkyl.

In another embodiment, L₁ and L₂ are each, independently, a bond or (C₁-C₁₀)alkylene.

In an embodiment, L₁ is a bond or (C₁-C₁₀)alkylene, and L₂ is (C₁-C₁₀)alkylene.

The divalent moiety D is a group having two points of connection, each point of connection represented by an asterisk.

The divalent moiety D is selected from the group consisting of

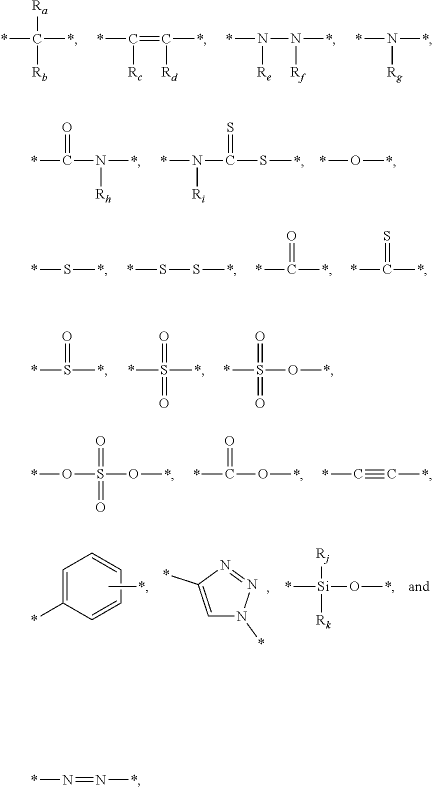

wherein each occurrence of Rₐ-Rₖ are each, independently, H, halogen, or hydrocarbyl.

In an embodiment, D is

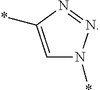 [SMILES: *c1cn(*)nn1]

Any of the substituents described herein may optionally be interrupted by one or more divalent moieties.

As used herein, the phrase “interrupted by one or more divalent moieties” when used in relation to a substituent means a modification to the substituent in which one or more divalent moieties are inserted into one or more covalent bonds between atoms. The interruption may be in a carbon-carbon bond, a carbon-hydrogen bond, a carbon-heteroatom bond, a hydrogen-heteroatom bond, or heteroatom-heteroatom bond. The interruption may be at any position in the substituent modified, even at the point of attachment to another structure.

In an embodiment, A is —COOR₇, —NR₈R₉, —PO₃R₁₀R₁₁, —CN, —SR₁₂ or —OR₁₆, wherein each occurrence of R₇, R₈, R₉, R₁₀, R₁₁ and R₁₂ are each H, and R₁₆ is aryl.

In another embodiment, R₁₆ is phenyl, typically substituted with CO₂H.

In yet another embodiment, the compound of formula (I) is represented by a structure selected from the group consisting of

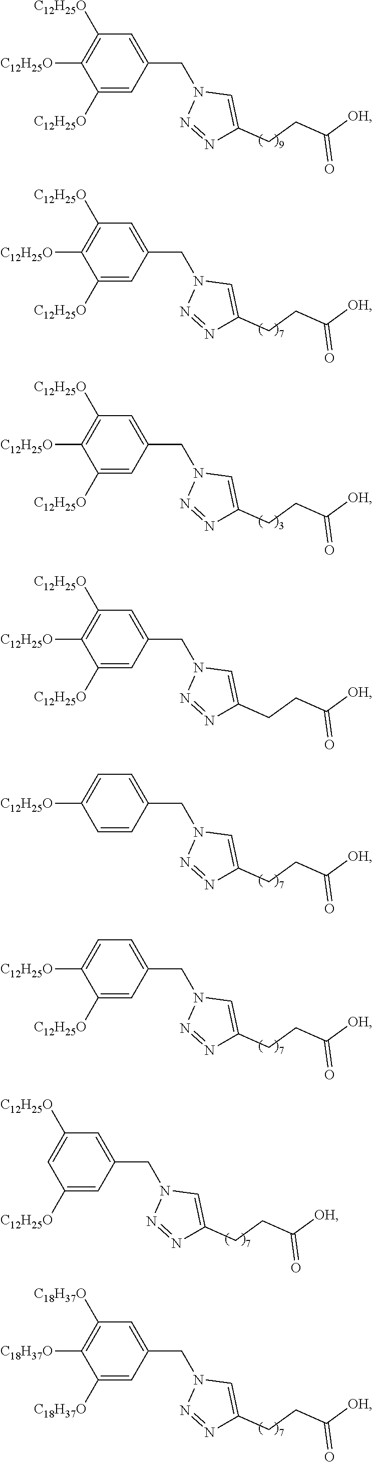

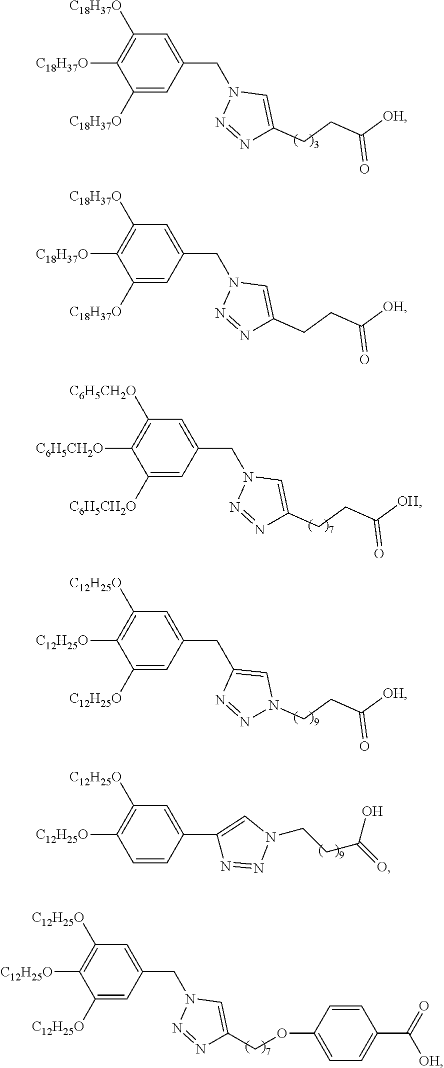

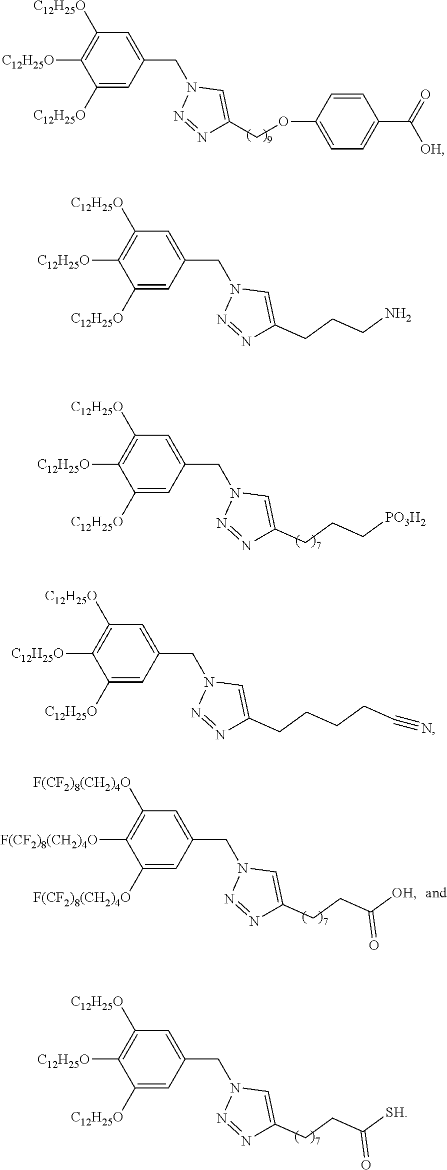

The present disclosure is also directed to a method for producing the compound represented by formula (I). The method comprises:
reacting a compound represented by the structure of formula (II):

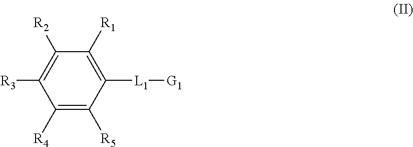

with a compound represented by the structure of formula (III):

G₂-L₂-A (III)
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
A is —COOR₇, —NR₈R₉, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl; and
each occurrence of G₁ is a reactive group capable of reacting with the reactive group G₂, and
G₂ is a reactive group capable of reacting with the reactive group G₁.

R₁-R₁₆, L₁, L₂, and A are as defined herein.

G₁ is a reactive group capable of reacting with the reactive group G₂, and G₂ is a reactive group capable of reacting with the reactive group in G₁.

In an embodiment, G₁ is a reactive group selected from the group consisting of —X, —NH₂, —N₃, —(C═O)X, -Ph(C═O)X, —SH, —CH═CH₂, and —C≡CH; wherein X is a leaving group.

In another embodiment, G₁ is —N₃ or —C≡CH.

In an embodiment, G₂ is a reactive group selected from the group consisting of —(C═O)X, —CH═CH₂, —C≡CH, —NH₂, —N₃, -Ph(C═O)X, —SH, —X, —NCO, —NCS; wherein X is a leaving group.

In another embodiment, G₂ is —N₃ or —C≡CH.

As used herein, the term “leaving group” refers to a molecular fragment that departs with a pair of electrons upon heterolytic bond cleavage. Leaving groups may be anions or neutral molecules and are known to those of ordinary-skill in the art. Suitable leaving groups include, but are not limited to, halides, such as, fluoride, chloride, bromide, and iodide; alkyl and aryl sulfonates, such as methanesulfonate (mesylate) and p-toluenesulfonate (tosylate); and hydroxide.

According to the present disclosure, it is understood that the reactive groups G₁ and G₂ may be reversed.

It would be understood by the ordinarily-skilled artisan that additional reagents may be needed to facilitate the reaction between the reactive groups G₁ and G₂, and that such additional reagents may be selected according to concepts well-known to those of ordinary skill in the chemical arts.

Any suitable reaction conditions, including reaction vessels and equipment, for the reacting step may also be selected by the ordinary-skilled artisan according to concepts known in the chemical arts.

The compounds represented by the structures of formulae (II) and (III) may be obtained from commercial sources or synthesized according to synthetic methods well-known to those of ordinary skill in the art.

For example, compounds represented by formula (II) may be synthesized according to Scheme 1, wherein n represents 0 or an integer greater than 0, typically 0 to 9. An ester may be reduced to the corresponding alcohol, and the resulting hydroxyl group is then converted to a better leaving group using known methods. For example, the hydroxyl group may be converted to a chloro group by using thionyl chloride (SOCl₂) reagent. The leaving group is then displaced by nucleophilic substitution, for example, by azide or acetylide.

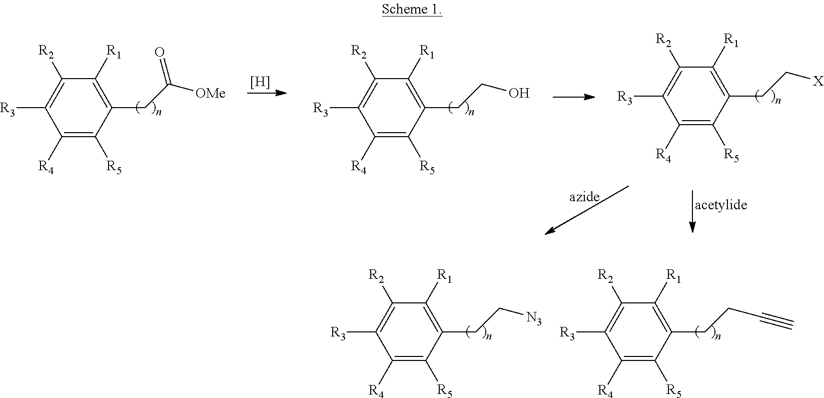

Compounds represented by formula (II) may also be synthesized according to Scheme 2. An aldehyde, such as the one shown in Scheme 2, may be converted, for example, into alkyne using known methods, such as Corey-Fuchs homologation.

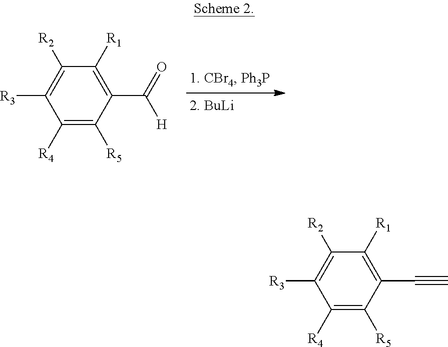

Suitable synthetic methods known to those of ordinary skill in the art are described in well-known texts, including, but not limited to, M. B. Smith “March's Advanced Organic Chemistry: Reactions, Mechanisms, and Structure”, 7^{th} edition (Wiley); and Carey and Sunberg “Advanced Organic Chemistry, Part A: Structure and Mechanisms”, 5^{th} edition (Springer) and “Advanced Organic Chemistry: Part B: Reaction and Synthesis”, 5^{th} edition (Springer).

The present disclosure also relates to a hybrid nanoparticle comprising:
(a) a metallic core, and
(b) a compound represented by formula (I) attached to the surface of the metallic core.

As used herein, the phrase “attached to the surface of the metallic core” in reference to the compound represented by formula (I) means that the compound represented by formula (I) may be connected to the metallic core by way of covalent bonding and/or non-convalent interaction between the metallic core and the compound represented by formula (I). Non-covalent interactions, as understood by those of ordinary skill in the art, include ionic bonds, dative bonds, hydrogen bonds, as well as Van der Waals interactions.

The metallic core is a metallic nanoparticle comprising or consisting of a metal, or an alloy or intermetallic comprising a metal. Metals include, for example, main group metals such as, e.g., lead, tin, bismuth, antimony and indium, and transition metals, e.g., a transition metal selected from the group consisting of gold, silver, copper, nickel, cobalt, palladium, platinum, iridium, osmium, rhodium, ruthenium, rhenium, vanadium, chromium, manganese, niobium, molybdenum, tungsten, tantalum, titanium, zirconium, zinc, mercury, yttrium, iron and cadmium.

The metallic nanoparticle may comprise metalloids and non-metals. Examples of non-metals include, but are not limited to, elements belonging to Group 13-17 of the Periodic Table of Elements, such as Group 15 elements, also known as pnictogens, such as phosphorus, and Group 16 elements, also known as chalcogens, such as oxygen, sulfur, selenium, and tellurium. The term “metalloid” refers to an element having chemical and/or physical properties intermediate of, or that are a mixture of, those of metals and nonmetals. Examples of metalloids include, but are not limited to, boron (B), silicon (Si), germanium (Ge), arsenic (As), and antimony (Sb). The metallic nanoparticle may also comprise f-block elements, typically understood to be the lanthanoids and actinoids on the Periodic Table of Elements. Examples of lanthanoids include, but are not limited to, lanthanum (La), cerium (Ce), europium (Eu), and erbium (Er), among others. Examples of actinoids include, but are not limited to, actinium (Ac), thorium (Th), uranium (U), and plutonium (Pu), among others.

In an embodiment, the metallic nanoparticle comprises or consists of oxides, phosphides, or chalcogenides of the metals described herein.

In an embodiment, the metallic core comprises indium, lead, a transition metal, or a mixture thereof.

In an embodiment, the metallic core comprises indium phosphide (InP).

In another embodiment, the metallic core comprises a lead chalcogenide, typically lead sulfide (PbS), lead selenide (PbSe), or lead telluride (PbTe).

In an embodiment, the metallic core comprises a lead chalcogenide, typically lead sulfide (PbS), lead selenide (PbSe), or lead telluride (PbTe).

In an embodiment, the metallic core comprises a transition metal oxide, typically zinc oxide (ZnO) or iron oxide (Fe₂O₃).

In an embodiment, the metallic core comprises a transition metal chalcogenide, typically cadmium sulfide (CdS), cadmium selenide (CdSe), or cadmium telluride (CdTe).

Herein, the term “nanoparticle” refers to a nano-sized structure, at least one dimension of which is less than or equal to 500 nm, typically less than or equal to 250 nm, more typically less than or equal to 100 nm, still more typically less than or equal to 50 nm. The metallic nanoparticles may be of any shape or geometry. For example, the nanoparticles described herein may be in the shape of cubes, rods, cylinders, spheres, polyhedrons, and the like. The nanoparticles may be amorphous or crystalline.

In an embodiment, the metallic core is a nanocrystal. As used herein, a nanocrystal is a nanoparticle comprising atoms having a highly-ordered crystalline arrangement. The nanocrystal may have monocrystalline or polycrystalline arrangement.

There is no particular limitation to the size of the metallic core of the hybrid nanoparticle described herein. Unless otherwise stated, the size of the metallic core of the hybrid nanoparticle is reported as a number average diameter, which may be determined using techniques and instrumentation known to those of ordinary skill in the art. For instance, transmission electron microscopy (TEM) may be used.

In an embodiment, the diameter of the metallic core is from about 1 nm to about 30 nm, typically from about 2 nm to about 15 nm, more typically from about 2 to about 10 nm.

TEM may be used to characterize size and size distribution, among other properties, of the nanoparticles. Generally, TEM works by passing an electron beam through a thin sample to form an image of the area covered by the electron beam with magnification high enough to observe the lattice structure of a crystal. The measurement sample is prepared by evaporating a solution or dispersion having a suitable concentration of the nanoparticles on a specially-made mesh grid, such as carbon-coated Cu TEM grids. The crystal quality of the nanoparticles can be measured by the electron diffraction pattern and the size and shape of the nanoparticles can be observed in the resulting micrograph image. Typically, the number of nanoparticles and projected two-dimensional area of every nanoparticle in the field-of-view of the image, or fields-of-view of multiple images of the same sample at different locations, are determined using image processing software, such as ImageJ (available from US National Institutes of Health). The projected two-dimensional area, A, of each nanoparticle measured is used to calculate its circular equivalent diameter, or area-equivalent diameter, x_{A}, which is defined as the diameter of a circle with the same area as the nanoparticle. The circular equivalent diameter is simply given by the equation

$${x}_{A} = \sqrt{\frac{4 A}{π}}$$

The arithmetic average of the circular equivalent diameters of all of the nanoparticles in the observed image is then calculated to arrive at the number average particle diameter, as used herein. A variety of TEM microscopes available, for instance, JEOL-1400 operated at 120 keV or JEOL 2100 TEM operating at 200 keV (available from JEOL USA). It is understood that all TE microscopes function on similar principles and when operated according to standard procedures, the results are interchangeable.

The present disclosure is directed to a method for producing a hybrid nanoparticle described herein, the method comprising:
forming the metallic core in the presence of a compound of formula (I); thereby producing the hybrid nanoparticle.

According to the present disclosure, producing the hybrid nanoparticle described herein is achieved by forming the metallic core in the presence of a compound of formula (I). Typically, one or more precursors to the metallic core and a compound of formula (I) are dissolved or dispersed in a solvent or solvent blend so as to obtain a reaction mixture. The temperature and/or pressure of the reaction mixture is then altered to those suitable for bringing about the formation of the metallic core with concomitant attachment of the compound of formula (I) to the surface of the metallic core.

Known efforts for producing nanoparticle-ligand hybrids rely on post-synthetic ligand-exchange, i.e., formation of the nanoparticle using a first ligand and then exchanging the first ligand with a second ligand at a later step after formation. In an embodiment, the method described herein does not comprise any post-synthetic ligand exchange step.

The present disclosure is directed to a film comprising a plurality of hybrid nanoparticles described herein.

The hybrid nanoparticles in the film may be the same or different. When the hybrid nanoparticles are the same, the hybrid nanoparticles are identical or substantially identical in the chemical nature of the metallic core, the compound represented by formula (I) attached to their surfaces, and their diameters. When the hybrid nanoparticles are different, the hybrid nanoparticles may differ in the chemical nature of the metallic core, the compound represented by formula (I) attached to their surfaces, and/or their diameters.

The hybrid nanoparticles described in the present disclosure may self-assemble into highly-ordered lattices having long-range order. Hybrid nanoparticles of two different types may form a binary superlattice. In an embodiment, the hybrid nanoparticles in the film are of two different types and form a binary superlattice.

In an embodiment, the binary superlattice is isostructural with NaZn₁₃, Cu₃Au, CuAu, MgZn₂, CaCu₅, or AlB₂ type lattice structures.

The number of hybrid nanoparticles in the binary superlattice is at least on the order of 10³ particles, typically at least on the order of 10⁴ particles, more typically at least on the order of 10⁵ particles, even more typically at least on the order of 10⁶ particles. In an embodiment, the number of hybrid nanoparticles in the binary superlattice is on the order of 10³ to 10⁶ particles.

The film described herein may be produced by any method known to those having ordinary skill in the art. Suitable film-forming techniques, include, but are not limited to vapor deposition, liquid deposition (continuous and discontinuous techniques), and thermal transfer. Continuous deposition techniques, include but are not limited to, casting, spin coating, gravure coating, curtain coating, dip coating, slot-die coating, spray coating, and continuous nozzle coating. Discontinuous techniques include, but are not limited to, ink jet printing, gravure printing, and screen printing.

In an embodiment, the film according to the present invention is formed by a method comprising:
(i) coating a composition comprising the hybrid nanoparticles described herein and a liquid carrier on the surface of a liquid immiscible with the liquid carrier of the composition, and
(ii) removing the liquid carrier of the composition, thereby producing the film.

The liquid carrier used in the composition comprises an organic solvent or a blend of organic solvents. In an embodiment, the composition consists essentially of or consists of an organic solvent or a blend of organic solvents. The blend of organic solvents comprises two or more organic solvents.

Organic solvents suitable for use in the liquid carrier may be polar or non-polar, protic or aprotic solvents. Examples of suitable organic solvents include, but are not limited to, chlorinated solvents, such as, for example, chloroform, carbon tetrachloride, and dichloromethane; alkane and alkene solvents, such as, for example, pentane, hexane, heptane, 1-octadecene, and isomers thereof; aromatic solvents, such as, for example, benzene, toluene, and tetralin (1,2,3,4-tetrahydronaphthalene); and alcohols, such as, for example, n-propanol, isopropanol, ethanol, and methanol, and alkylene glycol monoethers.

In an embodiment, the liquid carrier comprises hexane, or isomers thereof.

The amount of liquid carrier in the composition according to the present disclosure is from about 50 wt. % to about 99 wt. %, typically from about 75 wt. % to about 99 wt. %, still more typically from about 90 wt. % to about 99 wt. %, with respect to the total amount of composition.

The step of coating the composition described herein on the surface of a liquid immiscible with the liquid carrier of the composition may be achieved using any method known to the ordinarily-skilled artisan. For example, a drop of the composition may be spread on the surface of a liquid immiscible with the liquid carrier of the composition.

The liquid immiscible with the liquid carrier of the composition may be any solvent or blend of solvents that is immiscible with the liquid carrier of the composition. In an embodiment, the liquid immiscible with the liquid carrier of the composition is diethylene glycol.

Subsequent to the coating step, the step of removing the liquid carrier of the composition may be achieved according to any method known to the ordinarily-skilled artisan. For example, the liquid carrier of the composition may be allowed to evaporate under temperatures and pressures selected by the artisan based on the liquid carrier to be removed. In an embodiment, the step of removing the liquid carrier of the composition is carried out under ambient temperature and pressure.

The present disclosure is also directed to use of a compound represented by formula (I) for producing a hybrid nanoparticle comprising a metallic core.

The present disclosure is also directed to a method for making nanoparticles comprising a rare earth element, the method comprising:
(a) heating one or more reaction vessels, each said vessel containing a reaction mixture comprising a rare earth-containing precursor compound, and
(b) recovering the nanoparticles formed in the one or more reaction vessels in step (a).

The heating step, herein step (a), may be carried out using any heating source known to the ordinarily-skilled artisan. Suitable examples include, but are not limited to, heating mantles and heating baths, such as, for example, mineral oil bath, silicone oil bath, salt bath, and the like. Typically, a heating bath is used. The choice of material for the heating bath depends on the temperatures required and may be selected according to the standard methods known to those of ordinary skill in the art. In an embodiment, a salt bath is used. Typically, the salt bath is a 1:1 eutectic mixture of KNO₃:NaNO₃. A 1:1 eutectic mixture of KNO₃:NaNO₃ is advantageous because precise temperature control is accessible above the melting point of the salt, with a range of 160-500° C., and also because once the bath has reached the target temperature, the bath's temperature fluctuates by less than 0.5° C./min, mitigating batch-to-batch variation of the ramp rate and temperature profile.

In an embodiment, the salt bath, while molten, can be stirred to ensure even heating of the one or more reaction vessels.

The one or more reaction vessels may be heated by separate heat sources or by the same heat source. Heating by the same heat source allows the temperature profile between the one or more reaction vessels to be consistent. In an embodiment, the one or more reaction vessels are heated by the same heating source.

In step (a), the heating temperature is in the range of 160° C. to 500° C., typically 300° C. to 350° C., more typically 310° C. to 340° C.

The heating time is in the range of 1 min to 60 minutes, typically 10 minutes to 40 minutes.

Any reaction vessel may be used for the one or more reaction vessels as long as the reaction vessel material does not impede the reaction. Typically, glass reaction vessels are used. Generally, the one or more reaction vessels each have a volume equal to or less than 30 mL and are referred to herein as microreactors. Typically, the one or more reaction vessels each have a volume equal to or less than 20 mL, more typically equal to or less than 10 mL.

As described herein, one or more reaction vessels are heated during the heating step. In an embodiment, 2 or more reaction vessels are heated during the heating step. In another embodiment, 6 or more reaction vessels are heated, typically by the same heat source. This allows for tandem reactions to ensure that one or more parameters, such as temperature or pressure, of a series of reactions will be the same. This improves reproducibility and the efficiency of trend investigation. Changing the dimensions of the heat source and microreactors can enable 6 or more parallel microreactions.

The rare earth-containing precursor compound is a compound that comprises a rare earth element. As used herein, rare earth elements include the lanthanoids, also known as the lanthanides, as well as scandium and yttrium. Thus, rare earth elements include, but are not limited to, cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb) and yttrium (Y).

Suitable rare earth-containing precursor compounds include, but are not limited to, rare earth oxides, rare earth hydroxides, rare earth salts of inorganic and organic acids such as, for example, nitrates, nitrites, sulfates, halides (e.g., fluorides, chlorides, bromides and iodides), carbonates, phosphates, azides, borates (including fluoroborates and pyrazolylborates), sulfonates, carboxylates (such as, for example, formates, acetates, propionates, oxalates and citrates), and substituted carboxylates (including halogenocarboxylates such as, for example, trifluoroacetates, hydroxycarboxylates, and am inocarboxylates).

In an embodiment, the rare earth-containing precursor compound comprises terbium, dysprosium, lanthanum, europium, yttrium, erbium or a combination thereof.

Examples of specific rare-earth containing precursor compounds for use in the present invention include, but are not limited to, dysprosium oxide, yttrium oxide, lanthanum oxide, erbium oxide, europium oxide, thulium oxide, terbium oxide, dysprosium trifluoroacetate, yttrium trifluoroacetate, lanthanum trifluoroacetate, erbium trifluoroacetate, europium trifluoroacetate, terbium trifluoroacetate, and thulium trifluoroacetate.

The above compounds may be employed as such or optionally as their hydrates. The above compounds may also be employed as mixtures thereof.

The reaction mixture or mixtures may further comprise a compound represented by formula (I). Thus, in an embodiment, the method comprises heating one or more reaction vessels, each said vessel containing a reaction mixture comprising a rare earth-containing precursor compound and a compound represented by formula (I).

In addition to the rare earth-containing precursor compound, the reaction mixture or mixtures may further comprise compounds known to be useful in the synthesis of nanoparticles.

In an embodiment, the reaction mixture or mixtures further comprise oleic acid.

In an embodiment, the reaction mixture or mixtures further comprise a source of fluoride. Suitable sources of fluoride include, for example, alkali metal fluorides, such as LiF, NaF, KF, and CsF. In an embodiment, the reaction mixture or mixtures further comprise LiF.

In an embodiment, the molar ratio of the compound represented by formula (I), when present, relative to oleic acid, when present, is from 99:1 to 20:80, typically from 80:20 to 20:80, more typically 50:50 to 40:60, still more typically 50:50.

The reaction medium used in the reaction mixture or mixtures comprises an organic solvent or a blend of organic solvents. In an embodiment, the reaction medium consists essentially of or consists of an organic solvent or a blend of organic solvents. The blend of organic solvents comprises two or more organic solvents.

Organic solvents suitable for use in the reaction medium may be polar or non-polar, protic or aprotic solvents. Examples of suitable organic solvents include, but are not limited to, chlorinated solvents, such as, for example, chloroform, carbon tetrachloride, and dichloromethane; alkane and alkene solvents, such as, for example, pentane, hexane, heptane, 1-octadecene, and isomers thereof; aromatic solvents, such as, for example, benzene, toluene, and tetralin (1,2,3,4-tetrahydronaphthalene); and alcohols, such as, for example, n-propanol, isopropanol, ethanol, and methanol, and alkylene glycol monoethers.

In an embodiment, the reaction medium comprises 1-octadecene.

The recovery of the nanoparticles formed in step (a) is carried out in step (b). Any method of isolating the formed nanoparticles known to those of ordinary skill in the art may be used, for example, filtration, centrifugation, and the like.

The present disclosure is also directed to the nanoparticles obtained by the method described herein.

The compounds, nanoparticles, such as nanocrystals, methods, and processes according to the present disclosure are further illustrated by the following non-limiting examples.

### EXAMPLES

The reagents and materials used in the following examples, unless otherwise stated, were obtained from commercial sources or synthesized from commercially-available reagents and compounds. The reagents and materials used in the following examples are summarized below.

1-Octadecene (90%), oleylamine (80-90%), chloroauric acid (HAuCl₄.3H₂O; ACS reagent), oleylamine (80-90%), 1,2,3,4-tetrahedronapthalene (tetralin, 98+%), copper (II) sulfate pentahydrate (98+) and 2,6-Di-tert-butyl-4-methylpyridine (98%) were purchased from Acros. Oleic acid (90%), cadmium acetylacetonate (99.9%), 1,2-dodecanediol (90%), tributylphosphine (97%), trioctylphosphine (90%), selenium powder (99.99%), sulfur powder (99.99%), diphenylphosphine (97%), selenium pellets (99.999%), zinc acetate dehydrate (99.999%), iron(III) acetylacetonate (99.9%), oleylalcohol (60%), octyl ether (99%), tris(trimethylsilyl) phosphine (95%), lead oxide (99.9%), indium acetylacetonate (99.99%), tert-butyl amine borane complex (TBAB, 97%), 1-bromododecane (97%), 1-bromooctadecane (≥97), benzyl bromide (98%), methyl 3,4,5-trihydroxybenzoate (98%), NaN₃(≥99.5%), sodium L-ascorbate (≥99%), N-(4-pentynyl)phthalimide (97%) 17, 10-undecynylphosphonic acid (≥95%) 19, LiAlH₄ (95%) were purchased from Aldrich. Hept-6-ynenitrile 22 and SOCl₂ (98%) was purchased from TCI America. 4-(Dodecyloxy)benzoic acid (98%) and hept-6-ynoic acid (95%) 13 were purchased from Alfa Aesar. Methyl 3,4-dihydroxybenzoate (97%) was purchased from Accela ChemBio Product List. Methyl 3,5-dihydroxybenzoate was were purchased from Santa Cruz Biotechnology. Undec-10-ynoic acid (98%) 12 was purchased from Frinton Laboratories. 4-Pentynoic acid (≥97%) 14 was purchased from Chem-Impex. Solvents were ACS grade or higher. CH₂Cl₂ was dried over CaH₂ and freshly distilled before use. HAuCl₄.3H₂O was stored in a 4° C. refrigerator.

Dysprosium oxide (99.9%), yttrium oxide (99.9%), lanthanum oxide (99.9%), erbium oxide (99.9%), europium oxide (99.9%), terbium oxide (99.9%), and thulium oxide (99.9%) was purchased from GFS Chemicals. Lithium fluoride (99.9%) was purchased from Sigma-Aldrich. Trifluoroacetic acid (TFA, 99% biochemical grade), potassium nitrate and sodium nitrate were purchased from Fisher Scientific.

1,2-Bis(dodecyloxy)-4-ethynylbenzene 10 was prepared according to the procedure described in Gehringer, L., Bourgogne, C., Guillon, D. & Donnio, B. “Liquid-Crystalline Octopus Dendrimers: Block Molecules with Unusual Mesophase Morphologies” J. Am. Chem. Soc. 126, 3856-3867 (2004). Compound 10 was isolated as a white solid (3.2 g, 80%) ¹H NMR (CDCl₃) δ 7.06 (dd, J=8.2, 1.9 Hz, 1H), 6.99 (d, J=1.9 Hz, 1H), 6.79 (d, J=8.3 Hz, 1H), 4.05-3.92 (m, 4H), 2.98 (s, 1H), 1.81 (p, J=6.8 Hz, 4H), 1.50-1.42 (m, 4H), 1.37-1.25 (m, 32H), 0.88 (t, J=6.9 Hz, 6H); ¹³C NMR (CDCl₃) δ 150.23, 148.81, 125.65, 117.30, 114.21, 113.27, 84.12, 75.54, 69.41, 69.27, 32.08, 29.85, 29.82, 29.78, 29.77, 29.55, 29.52, 29.35, 29.32, 26.15, 22.85, 14.27.

Tridec-12-ynoic acid 11 was prepared according to the procedure described in Evans, A. B., Flügge, S., Jones, S., Knight, D. W. & Tan, W.-F. “A new synthesis of the F5 furan fatty acid and a first synthesis of the F6 furan fatty acid” Arkivoc 2008, 95 (2008).

11-Azidoundecanoic acid 15 was prepared according to the procedure described in Anderson, C. A., Taylor, P. G., Zeller, M. A. & Zimmerman, S. C. “Room Temperature, Copper-Catalyzed Amination of Bromonaphthyridines with Aqueous Ammonia” J. Org. Chem. 75, 4848-4851 (2010).

4-(Undec-10-yn-1-yloxy)benzoic acid 16 was prepared according to the procedure described in Zhang, L.-Y., Zhang, Q.-K. & Zhang, Y.-D. “Design, synthesis, and characterisation of symmetrical bent-core liquid crystalline dimers with diacetylene spacer” Liq. Cryst. 40, 1263-1273 (2013). Compound 16 was isolated as a white solid (5 g, 83%). ¹H NMR (CDCl₃) δ 8.06 (d, J=8.9 Hz, 2H), 6.93 (d, J=8.8 Hz, 2H), 4.02 (t, J=6.5 Hz, 2H), 2.19 (td, J=7.1, 2.7 Hz, 2H), 1.94 (t, J=2.6 Hz, 1H), 1.87-1.75 (m, 2H), 1.57-1.49 (m, 2H), 1.49-1.27 (m, 10H); ¹³C NMR (CDCl₃) δ 172.17, 163.82, 132.47, 121.53, 114.33, 84.88, 68.40, 68.23, 29.52, 29.42, 29.22, 29.16, 28.85, 28.60, 26.10, 18.54.

11-Bromoundec-1-yne 18 was prepared according to the procedure described in Neef, A. B. & Schultz, C. “Selective fluorescence labeling of lipids in living cells” Angew. Chem. Int. Ed. Engl. 48, 1498-500 (2009).

Unless otherwise stated, the general methods used in the following examples are summarized below.

Nuclear Magnetic Resonance (NMR) spectroscopy. ¹H NMR (500 MHz) and ¹³C NMR (126 MHz) spectra were recorded on Bruker UN1500 or BIODRX500 NMR spectrometer. ¹H and ¹³C chemical shifts (5) are reported in ppm while coupling constants (J) are reported in Hertz (Hz). The multiplicity of signals in ¹H NMR spectra is described as “s” (singlet), “d” (doublet), “t” (triplet), “q” (quartet),“p” (pentet), “dd” (doublet of doublets) and “m” (multiplet). All spectra were referenced using solvent residual signals (CDCl₃: ¹H, δ 7.27 ppm; ¹³C, δ 77.2 ppm). 2D experiments such as Heteronuclear Single Quantum Coherence (HSQC) and Heteronuclear Multiple Bond Coherence (HMBC) was used to confirm NMR peak assignments. Reaction progress was monitored by thin-layer chromatography using silica gel coated plates or ¹H NMR.

Mass Spectroscopy. Matrix-assisted laser desorption/ionization time of flight (MALDI-TOF) mass spectrometry was performed on Bruker Ultraflex III (Maldi-Tof-Tof) mass spectrometer using dithranol as matrix.

Thermal Analysis. Thermogravimetric analysis (TGA) was conducted using a TA Instruments SDT Q600. Samples were heated at 20° C./min under air to 600° C. Thermal transitions were determined on a TA Instruments Q2000 differential scanning calorimeter (DSC) equipped with a liquid nitrogen cooling system with 10° C./min heating and cooling rates.

X-Ray Diffraction. X-ray diffraction measurements were performed using a Rigaku Smartlab diffractometer operating at 40 kV and 30 mA. The X-ray line source was Cu K-α. Small-angle X-ray diffraction was performed using a Brucker Multi-Angle X-ray scattering system at 54 cm with Cu K-α X-rays. In the case of the iron oxide samples, the source wavelength leads to attenuation, reducing the signal-to-noise and signal-to-background ratios.

Neutron Diffraction. Neutron diffraction was conducted at the NIST National Center for Neutron Research using the nSOFT 10 m SANS instrument. SANS was collected by contrast matching of partially-deuterated toluene solvent.

Electron Microscopy. Transmission electron microscopy (TEM) was performed using a JEOL-1400 microscope operated at 120 keV or a JEOL 2100 TEM operating at 200 keV. Samples for routine characterization were prepared by dropcasting dilute dispersions of NCs directly on to carbon-coated Cu TEM grids.

Tilt tomography was collected using a Fishcione model 2040 dual axis tomography holder. 3D reconstructions were calculated using ImageJ and the TomoJ plug-in.

Optical Absorption Spectroscopy. Optical absorption spectra were collected on an Cary 5000 spectrometer at 2 nm spectral band width. Samples were dissolved in carbon tetrachloride (for PbS, PbSe), chloroform (for Au), or hexanes and spectra collected in 1 cm quartz cuvettes.

Infrared Absorption Spectroscopy. Infrared absorption spectroscopy was collected using a model 8700 Fourier-transform infrared spectroscopy (FT-IR, Thermo-Fisher).

### Example 1. Synthesis of End Groups

The steps used to synthesize the end groups 5a-5e used in the inventive compounds are summarized in Scheme 3, wherein A, B, C, and D refer to general procedures described herein.

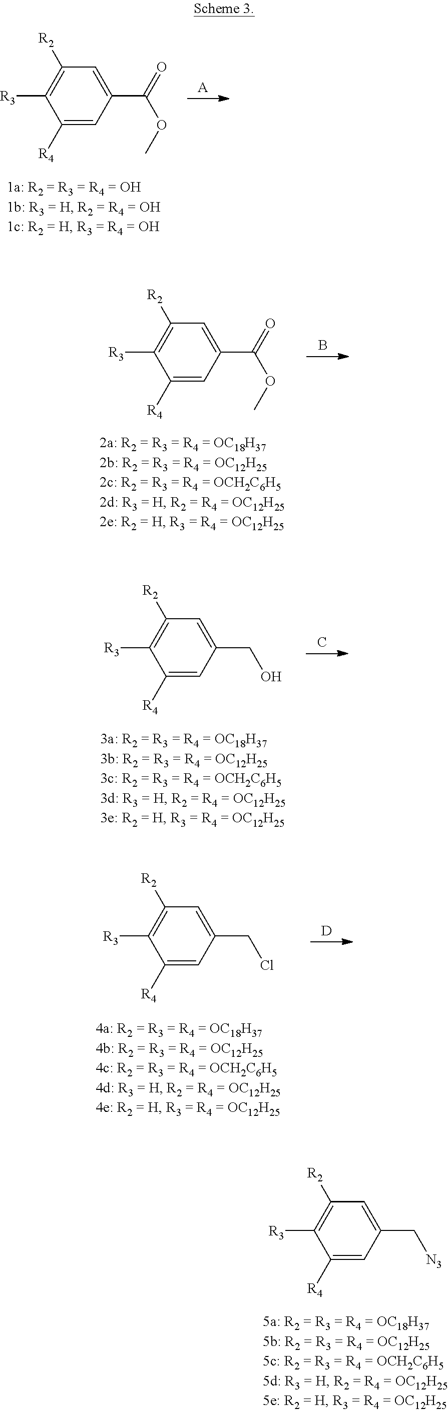

Methyl 3,4,5-tris(octadecyloxy)benzoate (Compound 2a) was prepared according to the following general procedure, designated general procedure A. To a stirred solution of methyl 3,4,5-trihydroxybenzoate 1a (5 g, 27.0 mmol) and 1-bromooctadecane (32.6 g, 97.8 mmol) in DMF (100 mL) was added K₂CO₃ (14.9 g, 108.0 mmol) and KI (0.45 g, 2.7 mmol) and the resulting mixture stirred at 90° C. for 12 h. The reaction mixture was cooled, diluted with CHCl₃ (200 mL), washed with H₂O (3×50 mL), dried over anhydrous MgSO₄, filtered and the filtrate concentrated under reduced pressure. The residue was redissolved in smallest possible amount of warm CHCl₃ and mixed with MeOH to induce the precipitation. The precipitate was collected by filtration and dried. The precipitation was repeated to obtain analytically pure methyl 3,4,5-tris(octadecyloxy)benzoate 2a (22.8 g, 90%). White solid. ¹H NMR (CDCl₃) δ 7.25 (s, 2H), 4.06-3.96 (m, 6H), 3.88 (s, 3H), 1.86-1.78 (m, 4H), 1.78-1.70 (m, 2H), 1.53-1.43 (m, 6H), 1.35-1.23 (m, 84H), 0.88 (t, J=6.9 Hz, 9H); ¹³C NMR (CDCl₃) δ 167.07, 152.96, 142.52, 124.79, 108.13, 73.62, 69.31, 52.22, 32.09, 30.49, 29.92, 29.90, 29.88, 29.83, 29.80, 29.73, 29.56, 29.53, 29.47, 26.24, 26.22, 22.85, 14.27.

Compounds 2b through 2e were synthesized according to general procedure A, except that methyl 3,4,5-trihydroxybenzoate and/or 1-bromooctadecane were replaced with other hydroxybenzoates and/or other haloalkanes.

Methyl 3,4,5-tris(dodecyloxy)benzoate 2b (24.7 g, 93%). White power. ¹H NMR (CDCl₃) δ 7.26 (s, 2H), 4.09-3.94 (m, 6H), 3.88 (s, 3H), 1.89-1.76 (m, 4H), 1.77-1.67 (m, 2H), 1.47 (p, J=7.2 Hz, 6H), 1.27 (s, 48H), 0.88 (t, J=6.7 Hz, 9H); ¹³C NMR (126 MHz, CDCl₃) δ 167.06, 152.95, 142.52, 124.78, 108.13, 73.61, 69.30, 52.21, 32.09, 32.08, 30.48, 29.90, 29.88, 29.87, 29.84, 29.81, 29.78, 29.72, 29.54, 29.51, 29.46, 26.23, 26.21, 22.84, 14.25.

Methyl 3,4,5-tris(benzyloxy)benzoate 2c (11.3 g, 81%). White solid. ¹H NMR (CDCl₃) δ 7.51-7.22 (m, 17H), 5.16 (s, 4H), 5.14 (s, 2H), 3.91 (s, 3H); ¹³C NMR (CDCl₃) δ 166.73, 152.68, 142.54, 137.57, 136.78, 128.66, 128.63, 128.30, 128.13, 128.05, 127.66, 125.34, 109.20, 77.41, 77.16, 76.91, 75.24, 71.35, 52.34.

Methyl 3,5-bis(dodecyloxy)benzoate 2d (7.7 g, 96%). White solid. ¹H NMR (CDCl₃) δ 7.16 (d, J=2.3 Hz, 2H), 6.63 (t, J=2.4 Hz, 1H), 3.96 (t, J=6.6 Hz, 4H), 3.89 (s, 3H), 1.84-1.71 (m, 4H), 1.55-1.39 (m, 4H), 1.39-1.21 (m, 32H), 0.88 (t, J=6.9 Hz, 6H); ¹³C NMR (CDCl₃) δ 167.09, 160.28, 131.92, 107.74, 106.68, 68.42, 52.25, 32.06, 29.80, 29.78, 29.74, 29.71, 29.51, 29.49, 29.32, 26.15, 22.83, 14.24.

Methyl 3,4-bis(dodecyloxy)benzoate 2e (8.3 g, 94%). White solid. ¹H NMR (CDCl₃) δ 7.63 (dd, J=8.4, 2.0 Hz, 1H), 7.54 (d, J=2.0 Hz, 1H), 6.86 (d, J=8.4 Hz, 1H), 4.10-3.98 (m, 4H), 3.88 (s, 3H), 1.88-1.77 (m, 4H), 1.52-1.43 (m, 4H), 1.39-1.22 (m, 32H), 0.88 (t, J=6.8 Hz, 6H); ¹³C NMR (CDCl₃) δ 167.10, 153.33, 148.64, 123.64, 122.53, 114.38, 112.05, 69.40, 69.13, 52.00, 32.07, 29.84, 29.83, 29.80, 29.77, 29.75, 29.55, 29.53, 29.51, 29.33, 29.22, 26.15, 26.11, 22.83, 14.24.

(3,4,5-Tris(octadecyloxy)phenyl)methanol (compound 3a) was synthesized according to the following general procedure, designated general procedure B. To a stirred solution of LiAlH₄ (1.09 g, 28.7 mmol) in dry THF at 0° C. was added methyl 3,4,5-tris(octadecyloxy)benzoate 2a (9.0 g, 9.6 mmol) portionwise over a period of 10 min and the resulting mixture stirred at 0° C. for 30 min under nitrogen atmosphere. It was then allowed to warm up to room temperature for 30 min after which the mixture was stirred at 60° C. for additional 3 h. The reaction mixture was cooled to 0° C. and quenched slowly by adding small portions of cold water while monitoring the evolution of hydrogen bubbles. The mixture was then concentrated under reduced pressure, dissolved in CHCl₃ (200 mL) and washed with 1N HCl (2×50 mL), dried over anhydrous Na₂SO₄, filtered and the filtrate concentrated under reduced pressure. The residue was redissolved in smallest possible amount of warm CHCl₃ and mixed with MeOH to induce the precipitation. The precipitate was collected by filtration and dried to obtain pure (3,4,5-tris(octadecyloxy)phenyl)methanol 3a (7.95 g, 91%). White solid. ¹H NMR (CDCl₃) δ 6.55 (s, 2H), 4.59 (s, 2H), 3.97 (t, J=6.5 Hz, 4H), 3.93 (t, J=6.6 Hz, 2H), 1.84-1.76 (m, 4H), 1.76-1.70 (m, 2H), 1.69 (s, 1H), 1.51-1.42 (m, 6H), 1.35-1.23 (m, 84H), 0.88 (t, J=6.9 Hz, 9H); ¹³C NMR (CDCl₃) δ 153.41, 137.73, 136.18, 105.48, 73.57, 69.25, 65.81, 32.09, 30.49, 29.92, 29.88, 29.82, 29.78, 29.58, 29.53, 26.30, 26.26, 22.85, 14.27.

Compounds 3b through 3e were synthesized according to general procedure B, except that compound 2a was replaced by compounds 2b-2e.

(3,4,5-tris(dodecyloxy)phenyl)methanol 3b (16.4 g, 95%). White power. ¹H NMR (CDCl₃) δ 6.54 (s, 2H), 4.58 (s, 2H), 3.96 (t, J=6.5 Hz, 4H), 3.93 (t, J=6.6 Hz, 2H), 1.82-1.71 (m, 7H), 1.52-1.40 (m, 6H), 1.36-1.23 (m, 48H), 0.88 (t, J=6.8 Hz, 9H); ¹³C NMR (126 MHz, CDCl₃) δ 153.36, 137.59, 136.19, 105.39, 73.58, 69.20, 65.76, 32.09, 32.07, 30.45, 29.90, 29.88, 29.85, 29.81, 29.79, 29.77, 29.56, 29.54, 29.51, 26.27, 26.24, 22.84, 14.26.

(3,4,5-Tris(benzyloxy)phenyl)methanol 3c (9.8 g, 86%). White solid. ¹H NMR (CDCl₃) δ 7.49-7.43 (m, 6H), 7.43-7.28 (m, 9H), 6.68 (s, 2H), 5.11 (s, 4H), 5.09 (s, 2H), 4.54 (s, 2H), 2.15 (s, 1H). ¹³C NMR (CDCl₃) δ 152.96, 137.88, 137.62, 137.16, 136.87, 128.66, 128.55, 128.22, 127.92, 127.88, 127.48, 106.31, 77.41, 77.16, 76.91, 75.31, 71.17, 65.25.

(3,5-Bis(dodecyloxy)phenyl)methanol 3d (6.1 g, 92%). White solid. ¹H NMR (CDCl₃) δ 6.49 (d, J=2.1 Hz, 2H), 6.37 (t, J=2.2 Hz, 1H), 4.59 (s, 2H), 3.92 (t, J=6.6 Hz, 4H), 1.94 (s, 1H), 1.81-1.72 (m, 4H), 1.49-1.39 (m, 4H), 1.36-1.23 (m, 32H), 0.89 (t, J=6.9 Hz, 6H); ¹³C NMR ((CDCl₃) δ 160.60, 143.32, 105.12, 100.60, 68.16, 65.49, 32.06, 29.81, 29.78, 29.75, 29.73, 29.54, 29.49, 29.39, 26.18, 22.83, 14.25.

(3,4-Bis(dodecyloxy)phenyl)methanol 3e (7.3 g, 91%). White solid. ¹H NMR (CDCl₃) δ 6.91 (s, 1H), 6.88-6.80 (m, 2H), 4.57 (s, 2H), 4.05-3.91 (m, 4H), 1.85-1.78 (m, 5H), 1.54-1.40 (m, 4H), 1.36-1.23 (m, 32H), 0.88 (t, J=6.9 Hz, 6H); ¹³C NMR (CDCl₃) δ 149.40, 148.76, 133.84, 119.67, 113.93, 113.05, 69.53, 69.32, 65.41, 32.05, 29.83, 29.79, 29.77, 29.57, 29.50, 29.43, 26.17, 26.15, 22.82, 14.24.

5-(Chloromethyl)-1,2,3-tris(octadecyloxy)benzene (compound 4a) was synthesized according to the following general procedure, designated general procedure C. To a stirred solution of (3,4,5-tris(octadecyloxy)phenyl)methanol 3a (5 g, 5.5 mmol) in dry CH₂Cl₂ (100 mL) was added DFM (0.05 mL) and thionyl chloride (1.95 g, 1.19 mL, 16.4 mmol) (for the synthesis of compound 4d, an equivalent amount (to thionyl chloride) of hindered base 2,6-di-tert-butyl-4-methylpyridine was also added) and the stirring continued for additional 3 h at room temperature under nitrogen atmosphere. The reaction mixture was then concentrated under reduced pressure and the residue redissolved in smallest possible amount of warm CHCl₃ and mixed with MeOH to induce the precipitation. The precipitate was collected by filtration and dried to obtain pure 5-(chloromethyl)-1,2,3-tris(octadecyloxy)benzene 4a (5.0 g, 98%). White solid.

¹H NMR (CDCl₃) δ 6.56 (s, 2H), 4.51 (s, 2H), 4.05-3.89 (m, 6H), 1.84-1.76 (m, 4H), 1.76-1.69 (m, 2H), 1.50-1.42 (m, 6H), 1.35-1.24 (m, 84H), 0.88 (t, J=7.0 Hz, 9H); ¹³C NMR (CDCl₃) δ 153.35, 138.45, 132.44, 107.21, 73.58, 69.28, 47.12, 32.09, 30.48, 29.91, 29.90, 29.87, 29.82, 29.80, 29.76, 29.57, 29.53, 26.27, 26.25, 22.85, 14.26.

Compounds 4b through 4e were synthesized according to general procedure C, except that compound 3a was replaced by compounds 3b-3e.

5-(Chloromethyl)-1,2,3-tris(dodecyloxy)benzene 4b (11.1 g, 99%). White power. ¹H NMR (CDCl₃) δ 6.57 (s, 2H), 4.51 (s, 2H), 4.02-3.89 (m, 6H), 1.84-1.76 (m, 4H), 1.76-1.69 (m, 2H), 1.52-1.42 (m, 6H), 1.36-1.23 (m, 48H), 0.89 (t, J=6.9 Hz, 9H); ¹³C NMR (CDCl₃) δ 153.32, 138.37, 132.44, 107.14, 77.41, 77.16, 76.91, 73.56, 69.22, 47.10, 32.09, 32.07, 30.46, 29.90, 29.88, 29.85, 29.81, 29.78, 29.75, 29.55, 29.51, 26.26, 26.23, 22.84, 14.25.

(((5-(Chloromethyl)benzene-1,2,3-triyl)tris(oxy))tris(methylene))tribenzene 4c (7.9 g, 98%). White solid. ¹H NMR (CDCl₃) δ 7.46-7.25 (m, 15H), 6.71 (s, 2H), 5.12 (s, 4H), 5.06 (s, 2H), 4.51 (s, 2H); ¹³C NMR (CDCl₃) δ 153.07, 138.72, 137.87, 137.02, 133.04, 128.68, 128.65, 128.30, 128.08, 127.96, 127.59, 108.43, 75.37, 71.43, 46.82.

Compound 4d was synthesized and taken to the next step without detailed purification.

4-(Chloromethyl)-1,2-bis(dodecyloxy)benzene 4e (5.8 g, 99%). White solid. ¹H NMR (CDCl₃) δ 6.96-6.86 (m, 2H), 6.82 (d, J=8.1 Hz, 1 H), 4.55 (s, 2H), 4.04-3.95 (m, 4H), 1.87-1.76 (m, 4H), 1.52-1.42 (m, 4H), 1.38-1.24 (m, 32H), 0.89 (t, J=6.8 Hz, 6H); ¹³C NMR (CDCl₃) δ 149.47, 149.34, 130.09, 121.36, 114.28, 113.53, 69.37, 69.35, 46.86, 32.06, 29.84, 29.80, 29.77, 29.56, 29.55, 29.51, 29.39, 29.36, 26.16, 26.15, 22.83, 14.25.

5-(Azidomethyl)-1,2,3-tris(octadecyloxy)benzene (compound 5a) was synthesized according to the following general procedure, designated general procedure D. To a solution of 5-(chloromethyl)-1,2,3-tris(octadecyloxy)benzene 4a (5 g, 5.4 mmol) in DMF (70 mL) was added NaN₃ (1.04 g, 16.1 mmol) and the resulting mixture was stirred at 90° C. for 36 h. The mixture was then cooled to 23° C., mixed with H₂O (100 mL) and extracted with CHCl₃ (3×100 mL). The organic layers were combined and washed with H₂O (2×100 mL), dried over anhydrous Na₂SO₄, filtered and the filtrate concentrated under reduced pressure. The residue was redissolved in smallest possible amount of warm CHCl₃ and mixed with MeOH to induce the precipitation. The precipitate was collected by filtration and dried to obtain pure 5-(azidomethyl)-1,2,3-tris(octadecyloxy)benzene 5a (4.78 g, 95%). White solid. ¹H NMR (CDCl₃) δ 6.49 (s, 2H), 4.24 (s, 2H), 4.05-3.91 (m, 6H), 1.85-1.76 (m, 4H), 1.76-1.69 (m, 2H), 1.51-1.43 (m, 6H), 1.36-1.24 (m, 84H), 0.88 (t, J=6.9 Hz, 9H); ¹³C NMR (CDCl₃) δ 153.50, 138.31, 130.48, 106.78, 77.41, 77.16, 76.90, 73.56, 69.33, 55.34, 32.09, 30.49, 29.92, 29.90, 29.88, 29.83, 29.81, 29.77, 29.58, 29.55, 29.53, 26.28, 26.25, 22.85, 14.27.

Compounds 5b through 5e were synthesized according to general procedure D, except that compound 4a was replaced by compounds 4b-4e.

5-(Azidomethyl)-1,2,3-tris(dodecyloxy)benzene 5b (9.2 g, 93%). White power. ¹H NMR (CDCl₃) δ 6.49 (s, 2H), 4.24 (s, 2H), 4.03-3.91 (m, 6H), 1.84-1.77 (m, 4H), 1.77-1.70 (m, 2H), 1.52-1.42 (m, 6H), 1.37-1.24 (m, 48H), 0.88 (t, J=6.8 Hz, 9H); ¹³C NMR (CDCl₃) δ 153.50, 138.31, 130.48, 106.78, 73.56, 69.32, 55.34, 32.10, 32.08, 30.49, 29.91, 29.89, 29.86, 29.82, 29.80, 29.77, 29.57, 29.55, 29.52, 26.28, 26.25, 22.85, 14.26.

Compounds 5c was synthesized and taken to the next step without detailed purification.

1-(Azidomethyl)-3,5-bis(dodecyloxy)benzene 5d (3.2 g, 91%). White solid. ¹H NMR (CDCl₃) δ 6.44 (d, J=1.9 Hz, 2H), 6.42 (d, J=2.0 Hz, 1H), 4.25 (s, 2H), 3.94 (t, J=6.6 Hz, 4H), 1.77 (p, J=6.7 Hz, 4H), 1.45 (p, J=6.9 Hz, 4H), 1.38-1.24 (m, 32H), 0.89 (t, J=6.9 Hz, 6H); ¹³C NMR (CDCl₃) δ 160.78, 137.53, 106.62, 101.24, 68.28, 55.10, 32.07, 29.82, 29.79, 29.75, 29.73, 29.54, 29.50, 29.39, 26.20, 22.84, 14.26.

4-(Azidomethyl)-1,2-bis(dodecyloxy)benzene 5e (4.1 g, 93%). White solid. ¹H NMR (CDCl₃) δ 6.92-6.78 (m, 3H), 4.24 (s, 2H), 4.06-3.94 (m, 4H), 1.88-1.79 (m, 4H), 1.52-1.43 (m, 4H), 1.38-1.24 (m, 32H), 0.89 (t, J=6.9 Hz, 6H); ¹³C NMR (126 MHz, CDCl₃) δ 149.43, 149.35, 127.90, 121.03, 113.96, 113.69, 69.37, 69.34, 54.88, 32.05, 29.83, 29.79, 29.76, 29.56, 29.50, 29.39, 26.16, 22.82, 14.23.

Steps used to synthesize end group 9 used in the inventive compounds are summarized in Scheme 4.

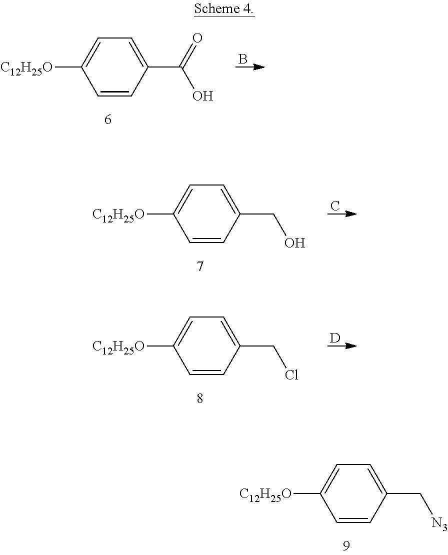

(4-(dodecyloxy)phenyl)methanol (compound 7) according to general procedure B except that the benzoate was replaced with compound 6, resulting in compound 7 as a white solid (10.1 g, 93%). ¹H NMR (CDCl₃) δ 7.27 (d, J=8.7 Hz, 2H), 6.88 (d, J=8.6 Hz, 2H), 4.59 (s, 2H), 3.95 (t, J=6.6 Hz, 2H), 1.83 (s, 1H), 1.82-1.70 (m, 2H), 1.52-1.40 (m, 2H), 1.40-1.20 (m, 16H), 0.89 (t, J=7.0 Hz, 3H); ¹³C NMR (126 MHz, CDCl₃) δ 158.90, 133.03, 128.73, 114.68, 68.20, 65.16, 32.05, 29.80, 29.77, 29.74, 29.72, 29.54, 29.48, 29.40, 26.17, 22.82, 14.25; MALDI-TOF (m/z): [M+Na]⁺ calcd. for C₁₉H₃₂O₂Na, 315.2300; found 315.188.

Compound 8 was synthesized according to general procedure C, except that compound 3a was replaced by compound 7. Compound 8 was taken to the next step without detailed purification.

Compound 9 synthesized according to general procedure D, except that compound 4a was replaced by compound 8. Compound 9 was taken to the next step without detailed purification.

### Example 2. Formation of Inventive Compounds

The Huisgen cycloaddition reaction was used to link the azide- or alkyne-functionalized end groups made according to Example 1 together with azide- or alkyne-functionalized surface anchoring groups, which were generally obtained from commercial sources unless otherwise stated, in the presence of a copper catalyst and sodium ascorbate.

The general procedure, designated general procedure E, for achieving the cycloaddition reaction is as follows. To a stirred solution of the azide (2.77 mmol), alkyne (3.3 mmol) and CuSO₄.5H₂O (0.21 g, 0.83 mmol) in THF/H₂O=4:1 (6 mL) was added sodium ascorbate (0.22 g, 1.11 mmol) and the resulting mixture stirred at 75° C. for 3 h under microwave irradiation (constant temperature mode). The solvent was evaporated, residue was dissolved in CHCl₃ (100 mL) and washed with 1N HCl (3×100 mL). The organic layer was dried over anhydrous Na₂SO₄, filtered and the filtrate concentrated under reduced pressure. The residue was redissolved in smallest possible amount of warm CHCl₃ and mixed with MeOH to induce the precipitation. The precipitate was collected by filtration and dried to obtain the inventive compound. The alkyne and azide reactants and the inventive compound formed therefrom are summarized in Table 1.

TABLE 1

| Inventive | | | |
|---|---|---|---|
| compound | Structure | Azide | Alkyne |
| 20a | 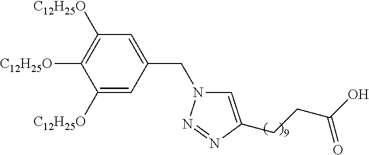 | 5b | 11 |
| 20b | 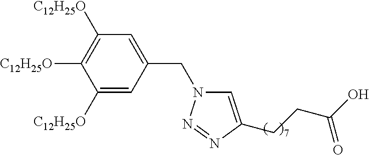 | 5b | 12 |
| 20c | 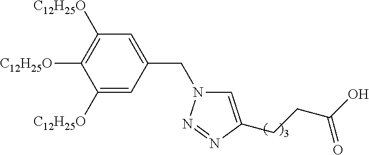 | 5b | 13 |
| 20d | 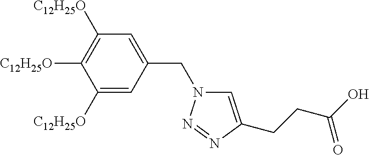 | 5b | 14 |
| 20e | 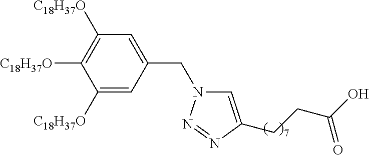 | 5a | 12 |
| 20f | 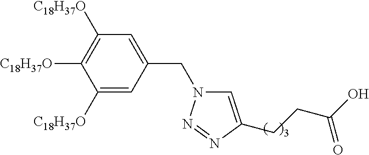 | 5a | 13 |
| 20g | 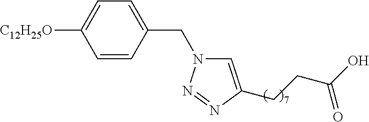 | 9 | 12 |
| 20h | 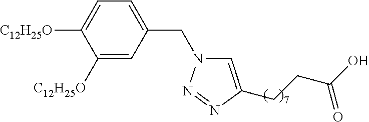 | 5e | 12 |
| 20i | 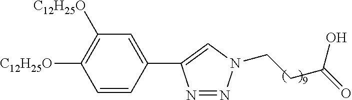 | 10 | 15 |
| 20j | 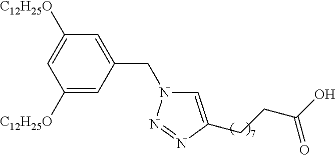 | 5d | 12 |
| 20k | 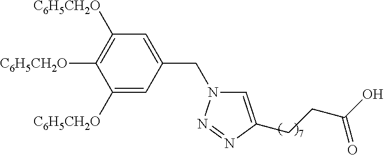 | 5c | 12 |
| 20l | 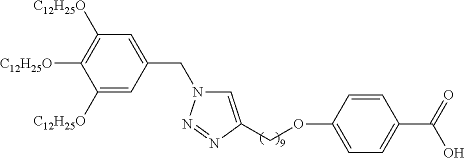 | 5b | 16 |
| 20m | 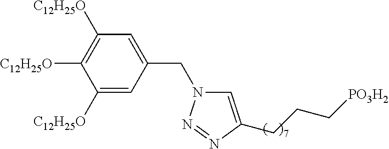 | 5b | 19 |
| 20n* | 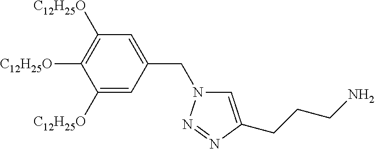 | 5b | 17 |
| 20o* | 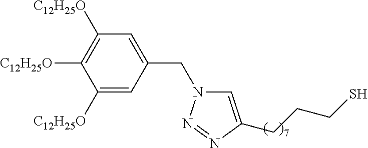 | 5b | 18 |
| 20p | 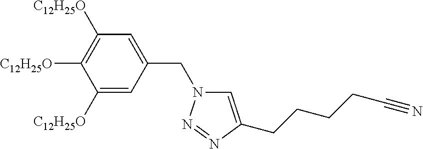 | 5b | 22 |

*required additional step following cycloaddition reaction

9-(1-(3,4,5-Tris(dodecyloxy)benzyl)-1H-1,2,3-triazol-4-yl)nonanoic acid 20a. (2.14 g, 89%). White solid. ¹H NMR (CDCl₃) δ 7.24 (s, 1H), 6.44 (s, 2H), 5.39 (s, 2H), 4.00-3.83 (m, 6H), 2.72 (t, J=6.8 Hz, 2H), 2.34 (t, J=7.4 Hz, 2H), 1.83-1.69 (m, 6H), 1.69-1.57 (m, 4H), 1.50-1.40 (m, 6H), 1.35-1.23 (m, 60H), 0.88 (t, J=7.0 Hz, 9H); ¹³C NMR (CDCl₃) δ 178.34, 153.77, 148.05, 138.75, 128.96, 121.23, 106.87, 73.63, 69.44, 55.08, 34.06, 32.09, 32.07, 30.46, 29.90, 29.88, 29.85, 29.84, 29.81, 29.79, 29.74, 29.57, 29.54, 29.51, 29.29, 29.21, 29.18, 29.12, 28.98, 26.25, 26.23, 25.28, 24.81, 22.84, 14.26. MALDI-TOF (m/z): [M+Na]⁺ calcd. for C₅₆H₁₀₁N₃O₅Na, 918.7639; found 919.060.

9-(1-(3,4,5-Tris(dodecyloxy)benzyl)-1H-1,2,3-triazol-4-yl)nonanoic acid 20b (4.2 g, 91%). White solid. ¹H NMR (CDCl₃) δ 7.18 (s, 1H), 6.43 (s, 2H), 5.36 (s, 2H), 3.98-3.85 (m, 6H), 2.68 (t, J=7.7 Hz, 2H), 2.33 (t, J=7.5 Hz, 2H), 1.81-1.69 (m, 6H), 1.68-1.56 (m, 4H), 1.49-1.39 (m, 6H), 1.35-1.23 (m, 56H), 0.87 (t, J=6.8 Hz, 9H); ¹³C NMR (CDCl₃) δ 178.90, 153.66, 138.53, 129.83, 120.65, 106.74, 73.60, 69.39, 54.50, 34.12, 32.08, 32.06, 30.45, 29.89, 29.87, 29.84, 29.80, 29.78, 29.74, 29.56, 29.53, 29.50, 29.25, 29.21, 29.19, 29.14, 26.25, 26.22, 25.72, 24.80, 22.83, 14.25; MALDI-TOF (m/z): [M+Na]⁺ calcd. for C₅₄H₉₇N₃O₅Na, 890.7326; found 890.994.

5-(1-(3,4,5-Tris(dodecyloxy)benzyl)-1H-1,2,3-triazol-4-yl)pentanoic acid 20c (1.5 g, 87%). White solid. ¹H NMR (CDCl₃) δ 8.80 (s, 1H), 7.39 (s, 1H), 6.46 (s, 2H), 5.39 (s, 2H), 4.01-3.81 (m, 6H), 2.75 (t, J=7.1 Hz, 2H), 2.36 (t, J=7.0 Hz, 2H), 1.82-1.61 (m, 10H), 1.49-1.40 (m, 6H), 1.35-1.21 (m, 48H), 0.86 (t, J=6.9 Hz, 9H); ¹³C NMR (CDCl₃) δ 177.93, 153.72, 147.26, 138.69, 128.96, 121.78, 106.93, 73.58, 69.38, 55.16, 33.74, 32.05, 32.03, 30.43, 29.86, 29.84, 29.82, 29.80, 29.77, 29.75, 29.71, 29.54, 29.50, 29.48, 28.52, 26.22, 26.21, 24.83, 24.25, 22.80, 14.22; MALDI-TOF (m/z): [M+Na]⁺ calcd. for C₅₀H₈₉N₃O₅Na, 834.6700; found 843.886.

3-(1-(3,4,5-Tris(dodecyloxy)benzyl)-1H-1,2,3-triazol-4-yl)propanoic acid 20d (1.8 g, 90%). White solid. ¹H NMR (CDCl₃) δ 7.31 (s, 1H), 6.43 (s, 2H), 5.36 (s, 2H), 3.98-3.83 (m, 6H), 3.13-2.93 (m, 2H), 2.85-2.69 (m, 2H), 1.85-1.64 (m, 6H), 1.54-1.39 (m, 6H), 1.36-1.22 (m, 48H), 0.88 (t, J=6.9 Hz, 9H); ¹³C NMR (CDCl₃) δ 176.79, 153.71, 146.77, 138.65, 129.45, 121.44, 106.83, 73.61, 69.41, 54.72, 33.44, 32.09, 32.07, 30.47, 29.90, 29.88, 29.85, 29.84, 29.81, 29.78, 29.75, 29.57, 29.54, 29.51, 26.25, 26.23, 22.84, 20.81, 14.26; MALDI-TOF (m/z): [M+Na]⁺ calcd. for C₄₈H₈₅N₃O₅Na, 806.6387; found 806.857.

9-(1-(3,4,5-Tris(octadecyloxy)benzyl)-1H-1,2,3-triazol-4-yl)nonanoic acid 20e (1.3 g, 94%). White solid. ¹H NMR (CDCl₃) δ 6.43 (s, 2H), 5.36 (s, 2H), 4.01-3.84 (m, 6H), 2.93-2.53 (m, 1H), 2.34 (t, J=7.4 Hz, 2H), 1.84-1.58 (m, 10H), 1.50-1.41 (m, 6H), 1.39-1.22 (m, 92H), 0.89 (t, J=6.9 Hz, 9H); ¹³C NMR (CDCl₃) δ 153.81, 139.16, 129.73, 107.33, 77.41, 77.16, 76.91, 73.70, 69.72, 54.81, 33.96, 32.09, 30.53, 29.90, 29.87, 29.81, 29.80, 29.76, 29.63, 29.60, 29.50, 29.25, 29.18, 29.15, 26.30, 26.28, 25.76, 24.86, 22.82, 14.18; MALDI-TOF (m/z): [M+Na]⁺ calcd. for C₇₂H₁₃₃N₃O₅Na, 1143.0143; found 1143.282.

5-(1-(3,4,5-Tris(octadecyloxy)benzyl)-1H-1,2,3-triazol-4-yl)pentanoic acid 20f (1.2 g, 92%). White solid. ¹H NMR (CDCl₃) δ 7.26 (s, 1H), 6.43 (s, 2H), 5.35 (s, 2H), 3.99-3.80 (m, 6H), 2.72 (s, 1H), 2.36 (t, J=6.7 Hz, 2H), 1.84-1.61 (m, 10H), 1.52-1.39 (m, 6H), 1.35-1.21 (m, 84H), 0.87 (t, J=6.9 Hz, 9H); ¹³C NMR (CDCl₃) δ 177.92, 153.65, 138.56, 129.57, 106.80, 73.58, 69.37, 54.75, 50.73, 33.79, 32.04, 30.44, 29.87, 29.84, 29.78, 29.77, 29.73, 29.55, 29.48, 28.66, 26.23, 26.21, 25.33, 24.36, 22.80, 14.22; MALDI-TOF (m/z): [M+Na]⁺ calcd. for C₆₈H₁₂₅N₃O₅Na, 1086.9517; found 1087.204.

9-(1-(4-(dodecyloxy)benzyl)-1H-1,2,3-triazol-4-yl)nonanoic acid 20g (2.1 g, 81%). White solid. ¹H NMR (500 MHz, Chloroform-d) δ 7.24-7.04 (m, 3H), 6.87 (d, J=7.6 Hz, 2H), 5.41 (s, 2H), 3.93 (t, J=6.0 Hz, 2H), 2.66 (s, 2H), 2.33 (t, J=7.0 Hz, 2H), 1.81-1.70 (m, 2H), 1.61 (s, 4H), 1.47-1.39 (m, 2H), 1.27 (d, J=18.7 Hz, 24H), 0.87 (t, J=6.1 Hz, 3H); ¹³C NMR (126 MHz, CDCl₃) δ 178.40, 159.77, 129.67, 126.90, 115.31, 68.45, 53.98, 34.14, 32.04, 29.77, 29.74, 29.71, 29.68, 29.50, 29.44, 29.41, 29.36, 29.24, 29.17, 29.15, 26.19, 25.74, 24.88, 22.77, 14.13; MALDI-TOF (m/z): [M+Na]⁺ calcd. for C₃₀H₄₉N₃O₃Na, 522.3672; found 522.859.

9-(1-(3,4-bis(dodecyloxy)benzyl)-1H-1,2,3-triazol-4-yl)nonanoic acid 20h (2.4 g, 85%). White solid. ¹H NMR (CDCl₃) δ 7.14 (s, 1H), 6.86-6.73 (m, 3H), 5.37 (s, 2H), 3.96 (t, J=6.6 Hz, 2H), 3.91 (t, J=6.5 Hz, 2H), 2.65 (t, J=7.6 Hz, 2H), 2.32 (t, J=7.5 Hz, 2H), 1.84-1.72 (m, 4H), 1.65-1.55 (m, 4H), 1.49-1.39 (m, 5H), 1.35-1.21 (m, 40H), 0.86 (t, J=6.8 Hz, 6H); ¹³C NMR (CDCl₃) δ 178.83, 149.58, 148.80, 127.21, 120.96, 120.51, 113.69, 69.42, 69.35, 54.09, 34.17, 32.03, 29.81, 29.80, 29.77, 29.73, 29.54, 29.52, 29.48, 29.42, 29.32, 29.21, 29.16, 29.10, 26.12, 25.67, 24.80, 22.80, 14.23; MALDI-TOF (m/z): [M+Na]⁺ calcd. for C₄₂H₇₃N₃O₄Na, 706.5499; found 706.676.

11-(4-(3,4-Bis(dodecyloxy)phenyl)-1H-1,2,3-triazol-1-yl)undecanoic acid 20i (1.8 g, 89%). White solid. ¹H NMR (CDCl₃) δ 7.67 (s, 1H), 7.47 (s, 1H), 7.32-7.16 (m, 1H), 6.90 (d, J=7.5 Hz, 1H), 4.36 (t, J=7.2 Hz, 2H), 4.07 (t, J=6.7 Hz, 2H), 4.01 (t, J=6.6 Hz, 2H), 2.33 (t, J=7.5 Hz, 2H), 2.01-1.88 (m, 2H), 1.88-1.70 (m, 4H), 1.62 (p, J=7.3 Hz, 2H), 1.54-1.41 (m, 4H), 1.41-1.10 (m, 44H), 0.87 (t, J=6.8 Hz, 6H); ¹³C NMR (CDCl₃) δ 178.78, 149.72, 149.36, 123.94, 118.38, 114.22, 111.56, 69.57, 69.51, 50.60, 34.04, 32.07, 30.45, 29.85, 29.81, 29.79, 29.79, 29.60, 29.51, 29.50, 29.47, 29.36, 29.33, 29.21, 29.10, 29.04, 26.59, 26.21, 26.19, 24.80, 22.83, 14.25; MALDI-TOF (m/z): [M+Na]⁺ calcd. for C₄₃H₇₅N₃O₄Na, 720.5655; found 720.705.

9-(1-(3,5-Bis(dodecyloxy)benzyl)-1H-1,2,3-triazol-4-yl)nonanoic acid 20j (0.4 g, 80%). White solid. ¹H NMR (CHCl₃) δ 7.22 (s, 1H), 6.38 (t, J=2.2 Hz, 1H), 6.35 (d, J=2.2 Hz, 2H), 5.36 (s, 2H), 3.86 (t, J=6.5 Hz, 4H), 2.67 (t, J=7.7 Hz, 2H), 2.31 (t, J=7.4 Hz, 2H), 1.72 (p, J=6.8 Hz, 4H), 1.66-1.54 (m, 4H), 1.47-1.36 (m, 4H), 1.36-1.20 (m, 40H), 0.86 (t, J=6.8 Hz, 6H); ¹³C NMR (CDCl₃) δ 178.96, 160.85, 148.68, 136.70, 120.86, 106.56, 101.32, 68.24, 54.35, 34.18, 31.98, 29.73, 29.70, 29.66, 29.63, 29.45, 29.41, 29.31, 29.26, 29.18, 29.15, 29.13, 29.08, 28.95, 28.71, 28.50, 26.08, 25.55, 24.78, 22.75, 18.44, 14.17; MALDI-TOF (m/z): [M+Na]⁺ calcd. for C₄₂H₇₃N₃O₄Na, 706.5499; found 706.657.

9-(1-(3,4,5-Tris(benzyloxy)benzyl)-1H-1,2,3-triazol-4-yl)nonanoic acid 20k (2.5 g, 77%). White solid. ¹H NMR (CDCl₃) δ 7.44-7.26 (m, 15H), 6.51 (s, 2H), 5.35 (s, 2H), 5.06 (s, 4H), 5.05 (s, 2H), 2.92-2.45 (m, 2H), 2.33 (t, J=7.4 Hz, 2H), 1.80-1.55 (m, 4H), 1.45-1.22 (m, 8H); ¹³C NMR (126 MHz, CDCl₃) δ 178.45, 153.24, 138.87, 137.74, 136.82, 130.28, 128.66, 128.31, 128.10, 128.02, 127.60, 108.07, 75.35, 71.45, 54.47, 34.03, 29.28, 29.20, 29.12, 25.78, 24.80; MALDI-TOF (m/z): [M+Na]⁺ calcd. for C₃₉H₄₃N₃O₅Na, 656.3100; found 656.478.

4-((9-(1-(3,4,5-Tris(dodecyloxy)benzyl)-1H-1,2,3-triazol-4-yl)nonyl)oxy)benzoic acid 20l (1.9 g, 87%). White solid. ¹H NMR (CDCl₃) δ 8.04 (d, J=8.8 Hz, 2H), 7.19 (s, 1H), 6.91 (d, J=8.8 Hz, 2H), 6.42 (s, 2H), 5.36 (s, 2H), 4.00 (t, J=6.5 Hz, 2H), 3.96-3.85 (m, 6H), 2.75-2.62 (m, 2H), 1.84-1.69 (m, 8H), 1.68-1.59 (m, 2H), 1.50-1.39 (m, 8H), 1.36-1.21 (m, 56H), 0.87 (t, J=6.9 Hz, 9H); ¹³C NMR (CDCl₃) δ 171.11, 163.65, 153.66, 138.51, 132.38, 129.84, 121.73, 114.27, 106.72, 73.61, 69.38, 68.36, 54.53, 32.06, 30.45, 29.88, 29.87, 29.84, 29.83, 29.80, 29.77, 29.73, 29.55, 29.52, 29.50, 29.45, 29.39, 29.34, 29.22, 26.24, 26.21, 26.08, 25.78, 22.83, 14.25; MALDI-TOF (m/z): [M+Na]⁺ calcd. for C₆₁H₁₀₃N₃O₆Na, 996.7745; found 997.031.

(9-(1-(3,4,5-Tris(dodecyloxy)benzyl)-1H-1,2,3-triazol-4-yl)nonyl)phosphonic acid 20m (0.5 g, 91%). White solid. ¹H NMR (CDCl₃) δ 7.26 (br s, 1H), 6.43 (s, 2H), 5.35 (s, 2H), 4.05-3.78 (m, 6H), 2.92-2.37 (m, 2H), 1.80-1.71 (m, 6H), 1.70-1.53 (m, 4H), 1.49-1.42 (m, 6H), 1.41-1.13 (m, 60H), 0.87 (t, J=6.7 Hz, 9H); ¹³C NMR (CDCl₃) δ 153.68, 153.49, 138.66, 130.47, 129.49, 106.88, 73.61, 69.41, 55.32, 32.07, 32.06, 30.47, 29.88, 29.87, 29.84, 29.83, 29.80, 29.78, 29.74, 29.57, 29.52, 29.50, 29.13, 28.99, 28.86, 26.24, 22.82, 14.23.

3-(1-(3,4,5-Tris(dodecyloxy)benzyl)-1H-1,2,3-triazol-4-yl)propan-1-amine 20n. Compounds 5b and 17 were reacted according to general procedure E to obtain 2-(3-(1-(3,4,5-Tris(dodecyloxy)benzyl)-1H-1,2,3-triazol-4-yl)propyl)isoindoline-1,3-dione, which is the phthalimide-protected version of compound 20n, as a white solid (1.5 g, 92%). Then, to a stirred solution of the phthalimide (2 g, 2.22 mmol) in THF (50 mL) was added hydrazine hydrate (0.17 g, 3.33 mmol) and the resulting mixture warmed to 60° C. After stirring for 4 h, the reaction mixture was cooled to room temperature and solvents removed under reduced pressure. The residue was dissolved in smallest possible amount of warm CHCl₃ and mixed with MeOH to induce the precipitation. The white precipitate was collected by filtration and dried to obtain pure 3-(1-(3,4,5-tris(dodecyloxy)benzyl)-1H-1,2,3-triazol-4-yl)propan-1-amine 20n (1.45 g, 85%). White solid. ¹H NMR (CDCl₃) δ 7.22 (s, 1H), 6.43 (s, 2H), 5.35 (s, 2H), 3.97-3.85 (m, 6H), 2.82-2.68 (m, 2H), 2.14-1.90 (m, 4H), 1.90-1.81 (m, 2H), 1.81-1.66 (m, 6H), 1.52-1.39 (m, 6H), 1.39-1.17 (m, 48H), 0.88 (t, J=6.8 Hz, 9H); ¹³C NMR (CDCl₃) δ 153.64, 148.06, 138.52, 129.79, 120.71, 106.73, 73.55, 69.34, 54.42, 41.33, 32.04, 32.03, 30.43, 29.85, 29.83, 29.81, 29.79, 29.76, 29.74, 29.70, 29.53, 29.49, 29.47, 26.22, 26.19, 23.08, 22.79, 14.21; MALDI-TOF (m/z): [M+Na]⁺ calcd. for C₄₈H₈₈N₄O₃Na, 791.6754; found 791.854.

9-(1-(3,4,5-Tris(dodecyloxy)benzyl)-1H-1,2,3-triazol-4-yl)nonane-1-thiol 20o. Compounds 5b and 18 were reacted according to general procedure E to obtain 4-(9-bromononyl)-1-(3,4,5-tris(dodecyloxy)benzyl)-1H-1,2,3-triazole, which was taken to the next step without detailed purification. To a stirred solution of 4-(9-bromononyl)-1-(3,4,5-tris(dodecyloxy)benzyl)-1H-1,2,3-triazole (0.3 g, 0.3 mmol) in ethanol (10 mL) was added thiourea (0.076 g, 1 mmol) and the resulting mixture stirred under reflux for 12 h. The reaction mixture was cooled to 23° C., basified using 2N NaOH (20 mL) and stirred for 20 min. The mixture was then acidified with 2N H₂SO₄ (30 mL), concentrated under reduced pressure and extracted with CHCl₃ (3×50 mL). Organic layer was dried over anhydrous Na₂SO₄, filtered and the filtrate concentrated. The residue was re-dissolved in smallest possible amount of warm CHCl₃ and mixed with MeOH to induce the precipitation. The white precipitate was collected by filtration and dried to obtain pure 9-(1-(3,4,5-tris(dodecyloxy)benzyl)-1H-1,2,3-triazol-4-yl)nonane-1-thiol 20o (0.26 g, 93%). White solid. ¹H NMR (CDCl₃) δ 7.18 (s, 1H), 6.42 (s, 2H), 5.36 (s, 2H), 3.97-3.86 (m, 6H), 2.75-2.57 (m, 4H), 1.81-1.68 (m, 6H), 1.68-1.54 (m, 4H), 1.44 (p, J=7.0 Hz, 6H), 1.36-1.23 (m, 58H), 0.88 (t, J=6.9 Hz, 9H); ¹³C NMR (CDCl₃) δ 153.70, 148.88, 138.60, 129.87, 120.64, 106.76, 73.61, 69.42, 54.50, 39.28, 32.09, 32.08, 30.48, 29.90, 29.88, 29.86, 29.84, 29.81, 29.79, 29.75, 29.57, 29.54, 29.52, 29.44, 29.40, 29.36, 29.24, 29.05, 28.66, 26.27, 26.23, 25.83, 22.84, 14.26; MALDI-TOF (m/z): [M+Na]⁺ calcd. for C₅₄H₉₉N₃O₃SNa, 892.7305; found 892.729.

5-(1-(3,4,5-Tris(dodecyloxy)benzyl)-1H-1,2,3-triazol-4-yl)pentanenitrile 20p (2.0 g, 90%). White solid. ¹H NMR (CDCl₃) δ 7.26 (s, 1H), 6.44 (s, 2H), 5.36 (s, 2H), 3.99-3.86 (m, 6H), 2.75 (s, 2H), 2.37 (t, J=6.8 Hz, 2H), 2.30-2.10 (m, 2H), 1.93-1.81 (m, 2H), 1.81-1.66 (m, 8H), 1.52-1.39 (m, 6H), 1.36-1.22 (m, 48H), 0.87 (t, J=6.9 Hz, 9H); ¹³C NMR (CDCl₃) δ 153.71, 138.69, 129.53, 119.60, 106.89, 73.60, 69.42, 54.75, 32.06, 32.04, 30.45, 29.87, 29.85, 29.82, 29.81, 29.78, 29.76, 29.72, 29.54, 29.50, 29.48, 28.32, 26.23, 26.21, 24.92, 24.85, 22.81, 17.08, 14.22. MALDI-TOF (m/z): [M+Na]+calcd. for C₅₀H₈₈N₄O₃Na, 815.6754; found 815.876.

### Example 3. Synthesis of CdS, CdSe, and CdTe Nanocrystals (NCs)

The syntheses of zinc blende CdS, CdSe, and CdTe were as follows.

For the synthesis of CdSe, a 25 ml 3-neck flask was loaded with 5 mL 1-octadecene (ODE), 0.4 mmol of a carboxylic acid-terminated compound (20a-l) made according to Example 2, 0.1 mmol Cd(acac)₂ and 0.05 mmol Se powder. The reaction was heated under vacuum to 120° C. and held for 1 hour, and then the flask was filled with nitrogen and heated to 240° C. and held 30 minutes. Upon heating, starting at ˜180° C., the reaction solution changed color from pale yellow or clear to yellow, then orange or red as the temperature increased. After 30 minutes, the reaction was allowed to cool to ˜70° C. and the product was isolated by precipitation with isopropanol (5× reaction volume). For particularly small particles, ethanol was also added to induce flocculation if isopropanol was insufficient. At least two further washing steps with chloroform/isopropanol were performed to remove excess unbound ligand. A similar version of this synthesis uses CdO instead of Cd(acac)₂, requiring heating to 250° C. to dissolve the CdO, then cooling to 100° C. whereupon Se powder can be added against nitrogen flow. This procedure gave indistinguishable results.

For CdTe NC synthesis, 0.06 mmol phosphonic acid terminated compound (20m) made according to Example 2, 0.03 mmol Cd(acac)₂ and 2.5 mL ODE were heated under vacuum to 120° C. in a 25 mL 3-neck flask and held for 1 hour. After flushing with nitrogen, 70 μL of an anhydrous solution of 1 M Se in tributylphosphine was injected into the reaction mixture. Then, the reaction was heated to 240° C. under nitrogen and held for 10 minutes, then allowed to cool to ˜60° C. when the CdTe NCs were isolated by precipitation with isopropanol. The NCs were subsequently washed with chloroform/isopropanol and hexanes/isopropanol mixtures.

### Example 4. Synthesis of ZnO NC

A 25 mL 3-neck flask was loaded with 0.5 mmol zinc acetate dehydrate, 1 mmol of carboxylic acid-terminated compound made according to Example 2, 2 mmol 1,2-dodecanediol, and 5 mL ODE. The reaction contents were dried under vacuum at 120° C. for 1 hr then heated under nitrogen flow to 300° C. For lower temperature decomposition of the zinc carboxylate precursor, monoalcohols or amines can be used, but the resulting size-uniformity of the particles was found to be lower. The NCs were purified after cooling to ˜60° C. by precipitation with acetone followed by two additional washing steps with chloroform/acetone and dispersed into hexanes. Later, NMR experiments were performed on samples after two additional washing steps with hexanes/isopropanol mixtures.

### Example 5. Synthesis of InP NC

A 25 mL 3-neck flask was loaded with 0.1 mmol In(acac)₃, 0.4 mmol carboxylic acid-terminated compound made according to Example 2, and 3 mL ODE. The flask was heated to 120° C. and held under vacuum 1 hour then heated under nitrogen to 240° C. Separately, a solution of 1.3 mL dry ODE, 200 μL oleylamine, and 0.05 mmol tris(trimethylsilyl)phosphine was prepared in a glovebox. This solution was injected into the reaction flask at 240° C., which turned orange-red immediately, and the reaction proceeded for 5 minutes before cooling by removing the heating mantle. The InP NCs were isolated by precipitation with ethanol and washed subsequently with chloroform/isopropanol mixtures.

### Example 6. Synthesis of PbS NC

0.2 mmol PbO, 0.4 mmol carboxylic acid-terminated compound made according to Example 2, and 3.5 mL of ODE were loaded into a 25 mL 3-neck flask and dried under vacuum for 1 hr at 130° C. After drying, under nitrogen, a solution of 2 mL dry ODE with 0.1 mmol bis(trimethylsilyl)sulfide was rapidly injected and the reaction was allowed to slowly cool to ˜50° C. The PbS NCs were precipitated by addition of ethanol and was twice more with chloroform/isopropanol mixtures and finally dispersed in hexanes.

### Example 7. Synthesis of PbSe NC

A 25 mL 3-neck flask was loaded with 0.2 mmol PbO, 0.4 mmol of a carboxylic acid-terminated ligand compound made according to Example 2, and 5 mL of ODE. The flask was held under vacuum at 120° C. for 1 hour to dissolve the PbO and form a clear solution. Then, under nitrogen, the flask was heated to 180° C. Separately, 400 μL of a solution containing 1 M Se pellets dissolved in trioctylphosphine was mixed with 15 μL of diphenylphosphine. This solution was rapidly injected into the reaction flask at 180° C. and the temperature reset to 160° C. and held for 20 minutes. After 20 minutes, the heating mantle was removed and the reaction pot allowed to cool to ˜50° C. and the product was isolated by precipitation with isopropanol. The isolated NCs were subsequently washed with two rounds of hexanes/isopropanol to remove excess unbound ligand. The resulting particles were ˜4.5 nm in size, although it is a general finding that results must be recalibrated for new batches of trioctylphosphine or diphenylphosphine. For the synthesis of larger particles, less diphenylphosphine should be used; for smaller, more.

### Example 8. Synthesis of Fe₂O₃ NC

A 25 mL 3-neck flask was loaded with 0.2 mmol Fe(acac)₃, 1 mmol carboxylic acid-terminated ligand compound made according to Example 2, 1 mL diphenyl ether, and 0.2 mL oleylalcohol. The flask was held under vacuum for 2 hrs at 120° C. Then, under nitrogen, the reaction was heated to 250° C. and held for 30 mins. The reaction was afterward allowed to cool to ˜60° C. and purified by precipitation with ethanol followed by three washing steps with chloroform/isopropanol and finally dispersed in hexanes.

### Example 9. Synthesis of Au NC

A 20 mL scintillation vial was loaded with 500 mg of amine-terminated ligand compound (20n) made according to Example 2, 1.5 mL 1,2,3,4-tetrahydronapthalene, and 1 mL chloroform, which was added to improve the solubility of the ligand. This mixture was stirred at 40° C. on a hotplate until becoming a homogeneous solution, after which 0.03 mmol of chloroauric acid (HAuCl₄) was added to the mixture and dissolved into a homogeneous orange solution via stirring. Separately, 8.6 mg of tert-butyl amine borane complex was dispersed by sonication in a mixture of 100 mg amine-terminated polycatenar ligand, 300 μL of 1,2,3,4-tetrahydronapthalene, and 400 μL of chloroform. From this solution, 400 μL was withdrawn and injected into the reaction vial containing gold precursor. The reaction was stirred at 40° C. for 1 hour. The Au NCs were isolated by precipitation with acetone, followed by three additional washes with chloroform/acetone mixtures and finally redispersed in chloroform.

### Example 10. Properties of Inventive Nanocrystals

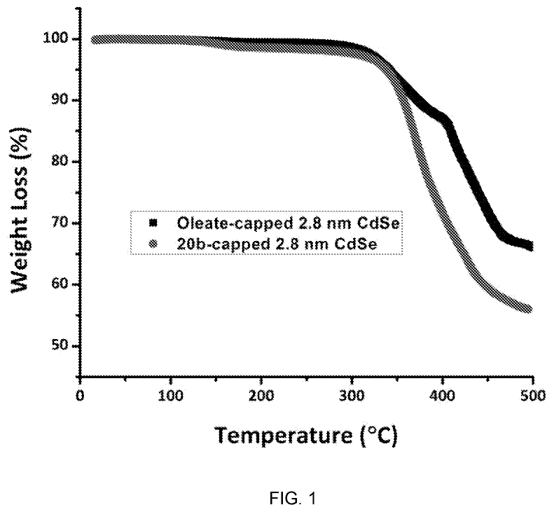

Thermal analysis of NCs coated with the inventive ligands made according to Example 2 indicates that decomposition of the organic material begins to occur at >300° C., similar to oleic acid. FIG. 1 shows the thermogravimetric analysis (TGA) of 20b-capped 2.8 nm CdSe NCs compared to oleate-capped 2.8 nm CdSe to under air flow. TGA analysis suggests that the ligand on the surface is considerably more massive than oleic acid, but such measurements are not chemically-specific.

To confirm that the NCs were terminated with the inventive polycatenar ligands described herein, FT-IR and NMR experiments were used. This is particularly a concern for those reactions, such as InP (oleylamine), Fe₂O₃ (oleyl alcohol), and ZnO (1,2-dodecanediol) in which other reagents may bind to the particle surface.

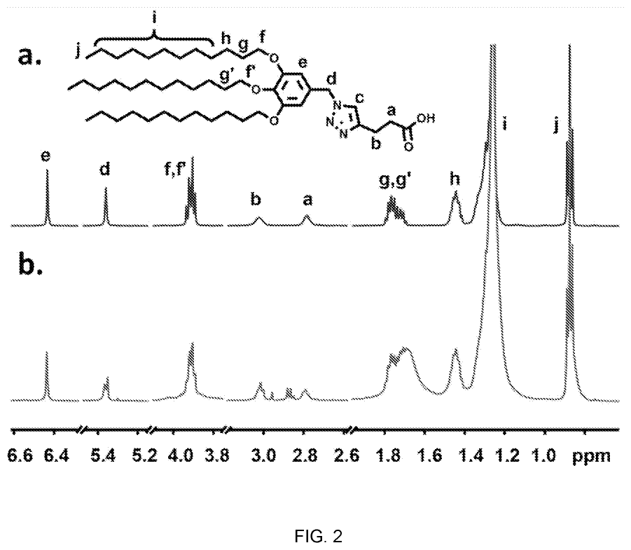

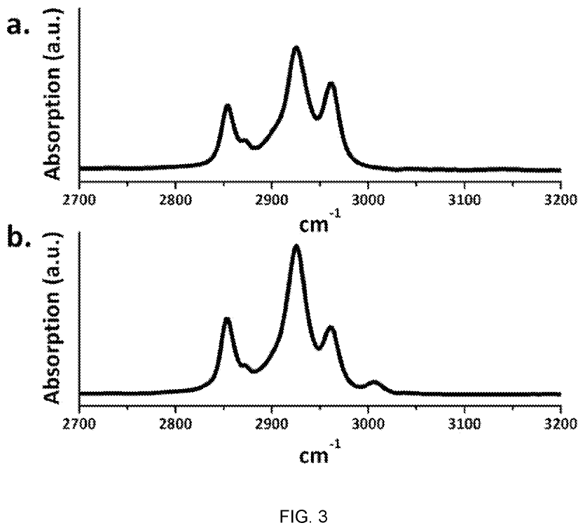

FIG. 2 shows (a) the proton NMR spectrum of ligand 20d and (b) NMR spectrum of 20d-capped 2.4 nm CdSe NCs showing the matching signals. The spectrum was taken after four precipitation steps to remove excess free ligand. FIG. 3 shows (a) the infrared absorption spectrum of 20d-capped CdSe NCs and (b) oleate-capped CdSe NCs. Alternatively, the presence of the inventive ligands may be verified by adding trimethylsilyl chloride to the solution of NCs in toluene, stripping ligands from the surface through the formation of (CH₃)₃Si—OR complexes and causing flocculation of the particles.

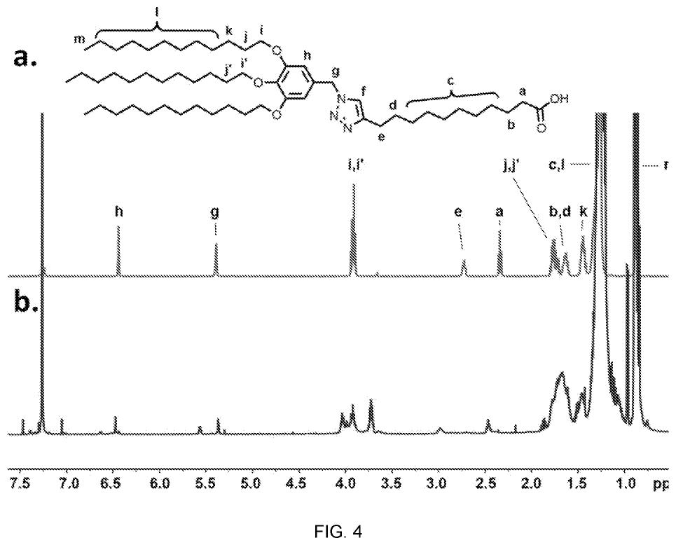

In those cases in which potentially non-innocent reagents were used, FT-IR confirmed that only in the case of ZnO was there a substantial amount of diol, which was used to induce decomposition of the metal carboxylate precursor, attached to the particles after several washing steps. Nonetheless, NMR experiments after three and five washing steps confirmed the persistent presence of polycatenar ligands, unambiguously identified by the presence of the aromatic protons. FIG. 4 shows (a) ¹H NMR spectra of polycatenar ligand 20a with corresponding signal assignments and (b) 20a-capped ZnO nanocrystals. Although some ligand bound to the surface is likely 1,2-dodecanediol (or a decomposition product), the presence of aromatic signatures from the ligand are still clear.

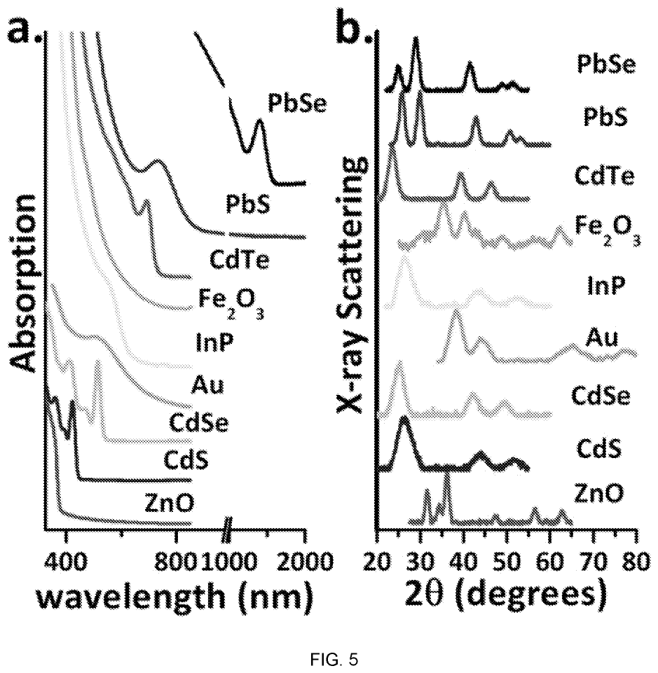

Visible and near-IR absorption spectra as well as X-ray diffraction were used to further characterize the inventive nanocrystals. FIG. 5 shows (a) the visible and near-IR absorption spectra and (b) the X-ray diffraction patterns for typical examples of each material. Au NCs show an fcc (face-centered cubic) structure. ZnO NCs exhibit the wurtzite crystal structure whereas CdS, CdSe, CdTe, and InP exhibit zinc blende crystal structures. Lead chalcogenides form in the rock-salt structure. Each of these crystal structures is the stable polymorph known for the given materials at the reaction temperatures used in the present examples. The X-ray structures of maghemite (Fe₂O₃) and magnetite (Fe₃O₄) are not sufficiently different for phase assignment, particularly when using a Cu K-α X-rays.

CdSe was chosen as a pilot synthesis to explore the influence of specific changes in the inventive polycatenar ligands because the size and monodispersity are easily evaluated on the basis of optical absorption data. Depending on the polycatenar ligand used, under the same reaction conditions, the resulting NCs varied in size from 2.2 nm to 6 nm. The first variable which was examined was the length of the surface anchoring group, which was varied from 5 to 13 carbons. As this linker increased in length, the particle size obtained in the synthesis also increased. Second, the dependence of the nanocrystal synthesis on the length of the terminating group was examined using C₁₂ and C₁₈ units. C₁₈-termination resulted in the largest particles obtained from any of the reactions with polycatenar ligands.

The alkyne and azide functionalities were reversed to test the stability of the ligands depending on the chemical route of linkage. No substantial influence of this change on the stability of the ligand under the tested reaction conditions was observed. Neither does there appear to be a problem with the presence or absence of a carbon group alpha to the aromatic rings.

Reactions with ligands 20h and 20j, with distinct peripheral chain substitution, produce soluble CdSe NCs with an average size consistent with the trend of steric hindrance (reactions with 3,5-branched 20h produced smaller NCs than with 3,4-branched 20j) but insufficiently monodisperse to demonstrate long-range self-assembly. Monoalkyl (20g), triphenyl (20k), and benzoate-anchoring (20l) derivatives showed poor solubility in situ and resulted in polydisperse, frequently aggregated products.

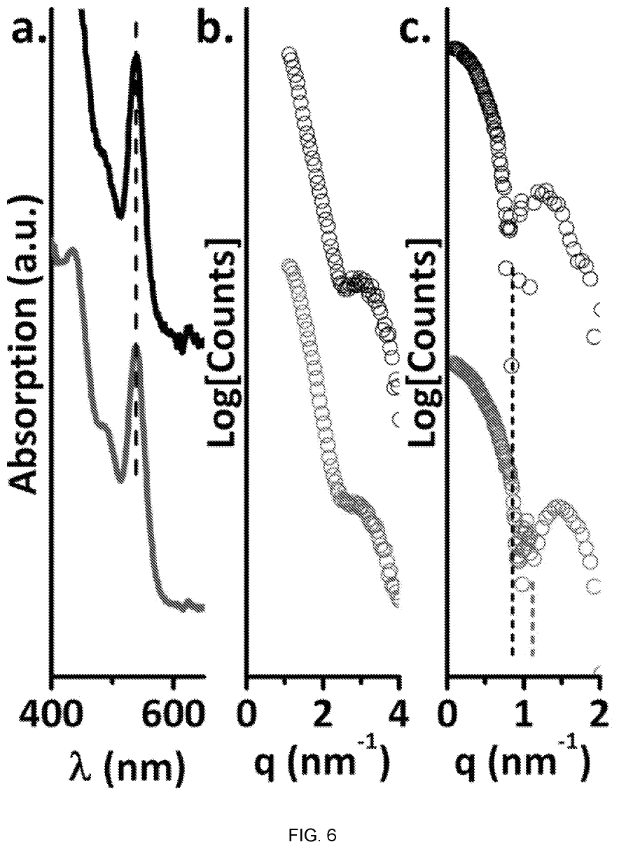

FIG. 6 shows (a) the optical absorption data, (b) SAXS data, and (c) SANS data for 20a-capped CdSe (bottom curve) and oleate-capped CdSe (top curve) NCs. Both samples were the same size, 2.8 nm. The data from SANS shows that the solvated polycatenar ligand coating is larger than the oleate ligand coating.

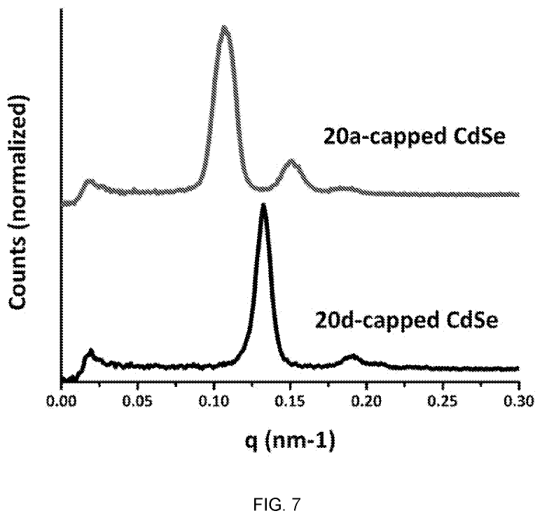

FIG. 7 shows the small-angle x-ray scattering from 20a- and 20d-capped CdSe NCs prepared by drop-casting on to a quartz coverslip. The observed peaks are consistent with bcc (body-centered cubic) ordering.

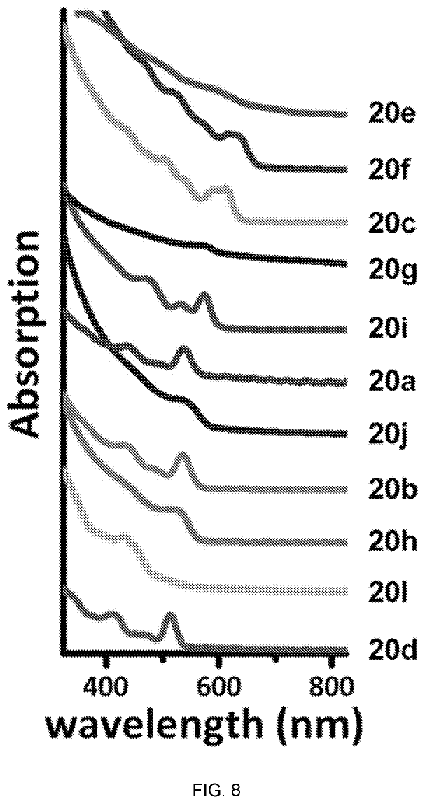

FIG. 8 shows the optical absorption spectra of several CdSe NC syntheses using the ligands 20a-20j and 20l.

### Example 11. Self-Assembly of NCs

In general, self-assembly of the inventive NCs was achieved as follows: a mixture containing one or two types of NC was prepared in hexanes at a defined stoichiometry and concentration (typically 5-10 mg/mL). After drying, the NCs were redispersed in 18 pL of hexanes and cast on to a diethylene glycol (DEG) surface formed by loading 1.7 mL diethylene glycol into 1.5 cm²×1.0 cm deep well machined from Teflon. The evaporating droplet was covered immediately with a glass slide to slow evaporation, which was typically complete after 1 hour. Once dry, the floating film was transferred to TEM grids by scooping up sections from below. Residual diethylene glycol was removed under vacuum before imaging.

Samples prepared by dropcasting dilute dispersions of NCs directly on to carbon-coated Cu TEM grids also resulted in self-assembly of the inventive NCs.

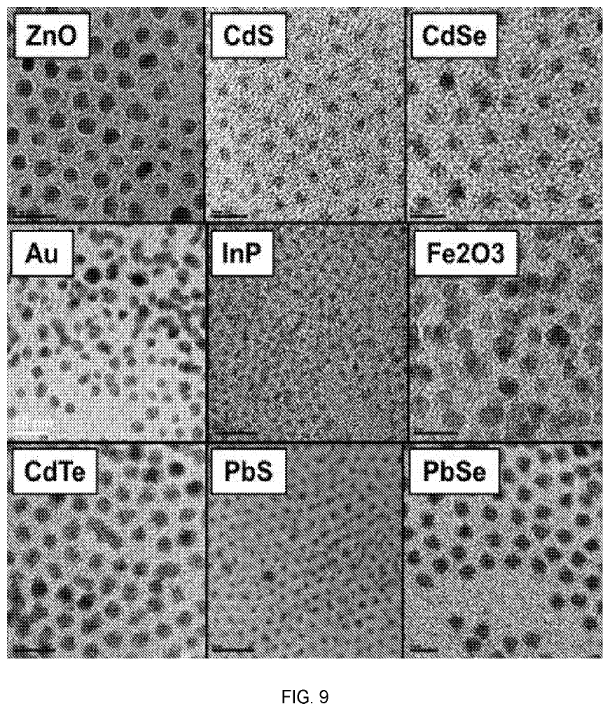

FIG. 9 shows the TEM Micrographs of colloidal nanocrystals synthesized the inventive ligands described herein, labeled by the inorganic material.

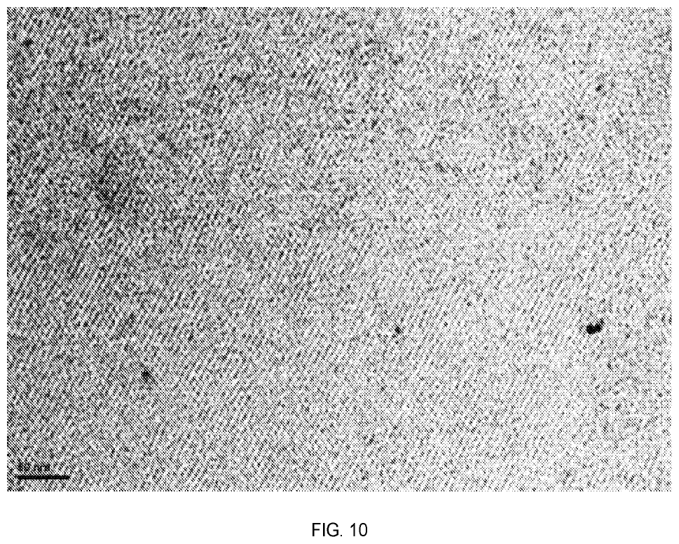

FIG. 10 shows 20b-capped 2.8 nm PbS NCs self-assembled on DEG into a bcc structure.

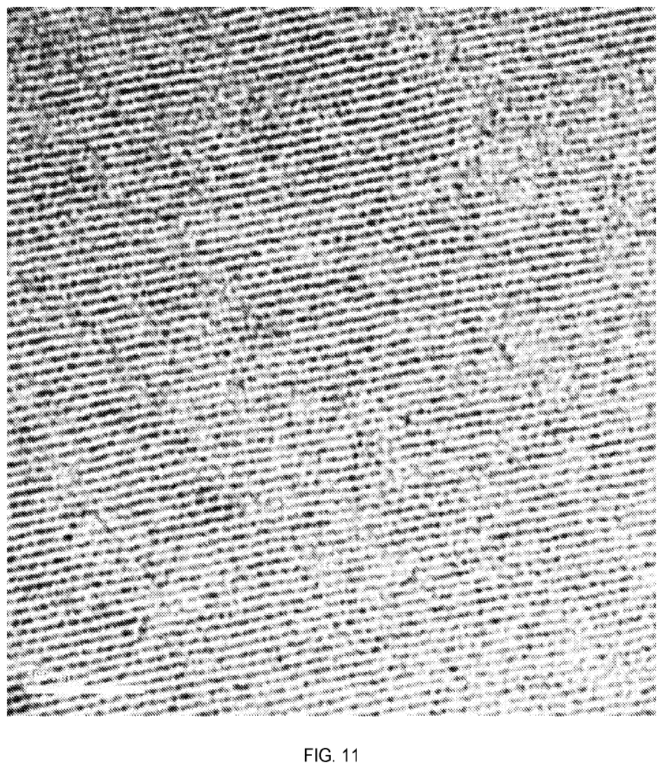

FIG. 11 shows 20b-capped 3.5 nm PbSe NCs self-assembled on DEG into a bcc structure.

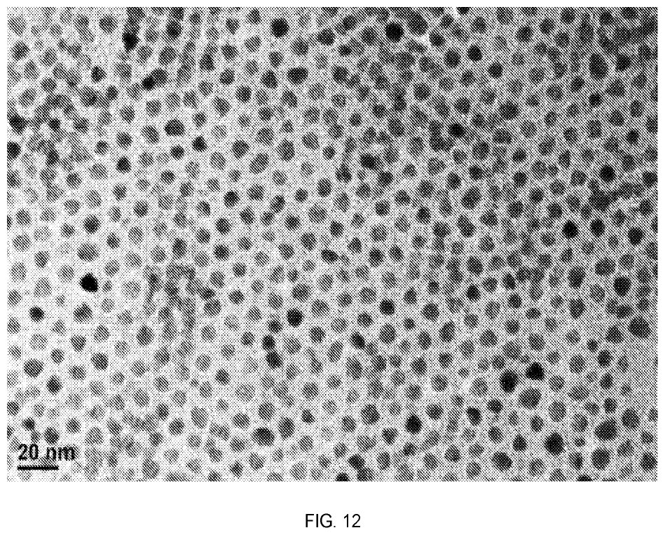

FIG. 12 shows 20i-capped 7.0 nm ZnO NCs drop-cast on to a TEM grid.

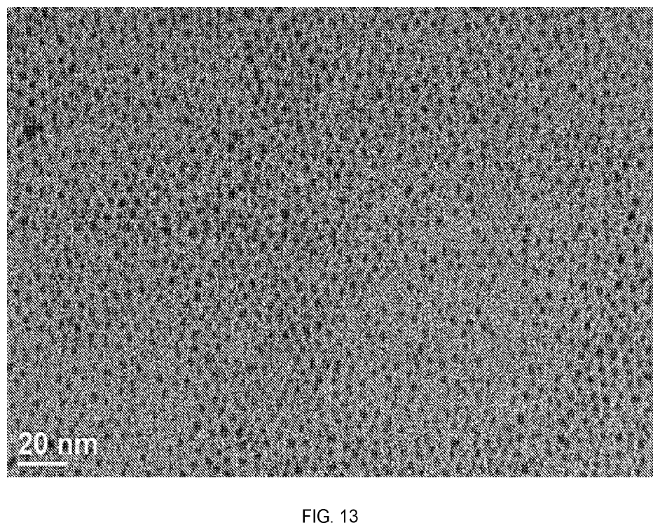

FIG. 13 shows 20n-capped 2.5 nm Au NCs drop-cast on a TEM grid.

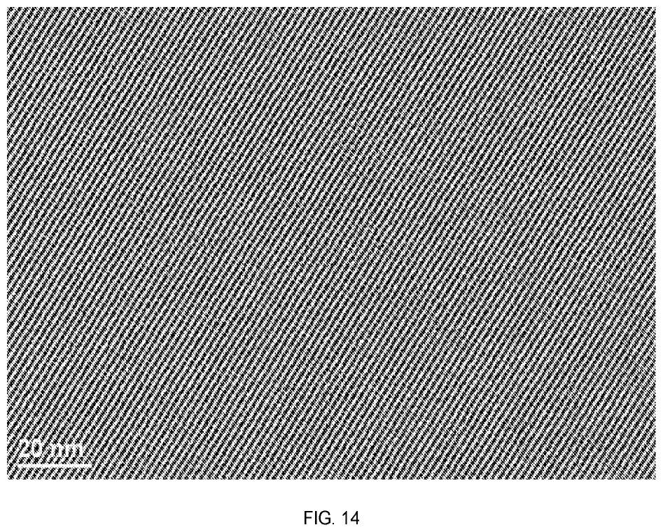

FIG. 14 shows 20d-capped 2.4 nm CdSe NCs self-assembled into a bcc superlattice.

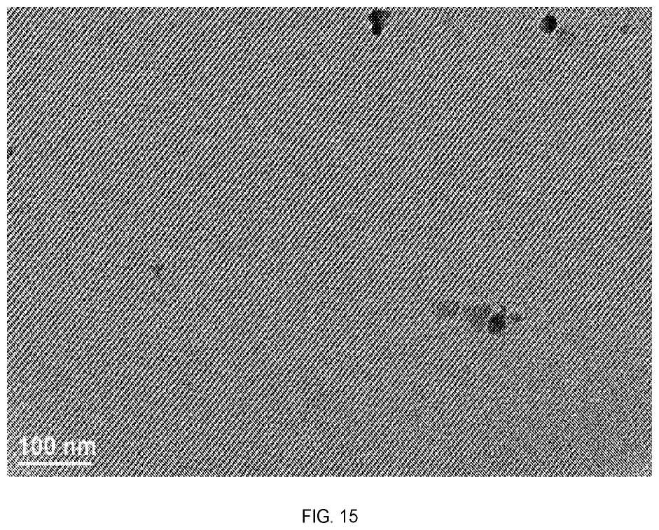

FIG. 15 shows 20b-capped 2.6 nm CdSe NCs self-assembled into a bcc superlattice.

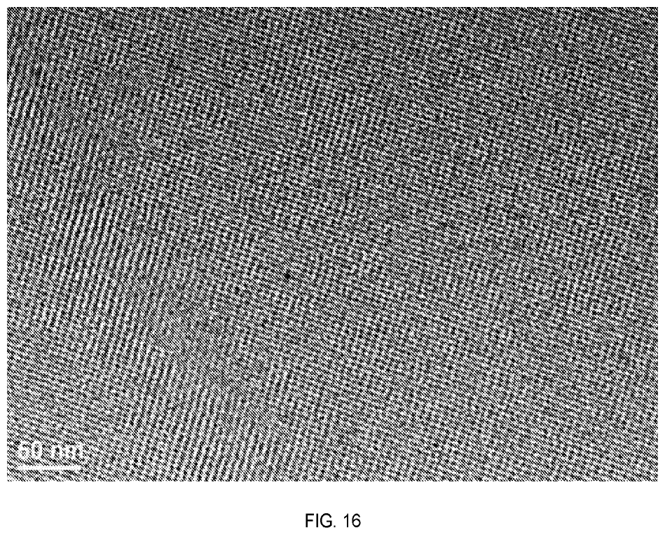

FIG. 16 shows 20a-capped 2.8 nm CdSe NCs self-assembled into a bcc superlattice.

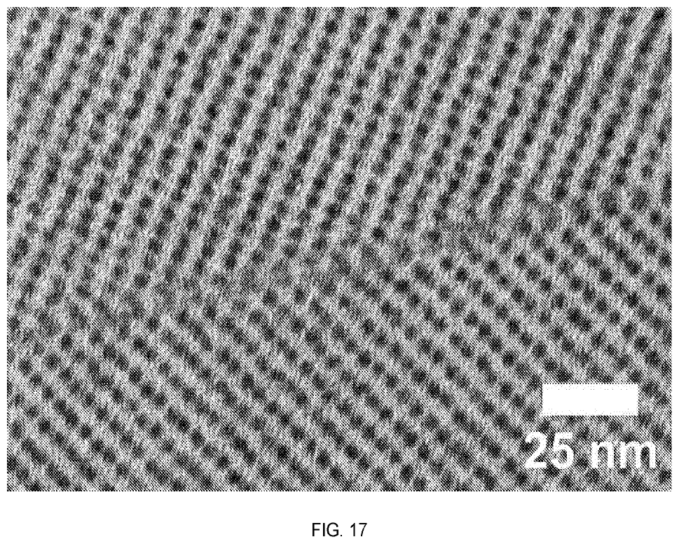

FIG. 17 shows the characteristic twin boundary of bcc superlattice composed of 20b-capped CdS NCs.

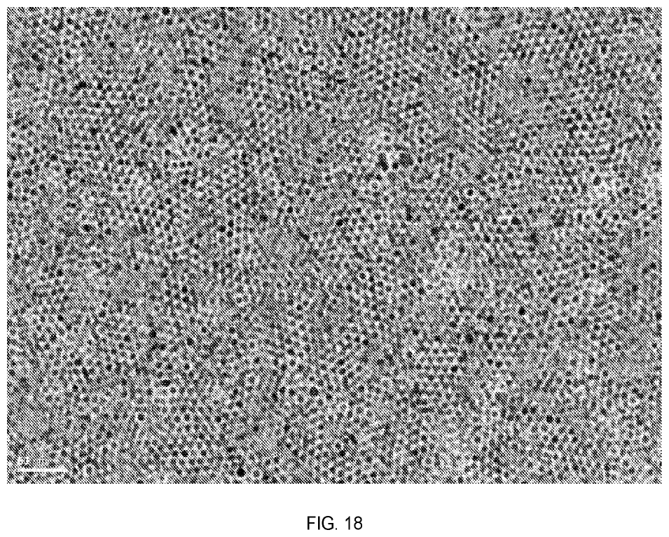

FIG. 18 shows 20c-capped CdSe forming short-range hcp lattices after drop-casting.

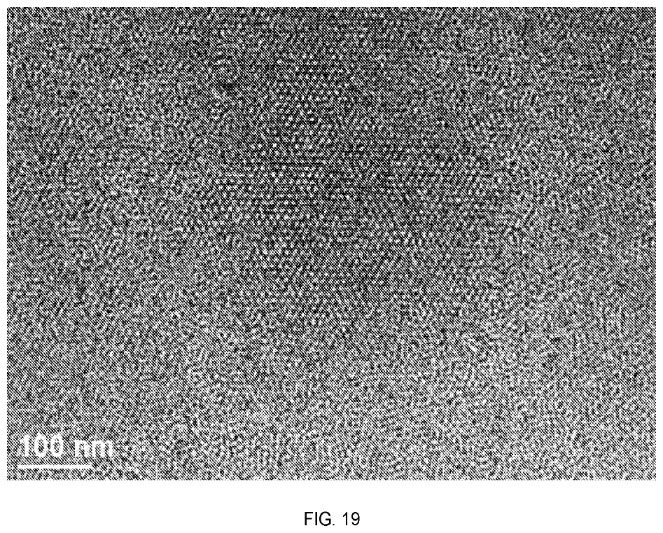

FIG. 19 shows the hcp (hexagonal close-packed) domain of 20f-capped CdSe NCs.

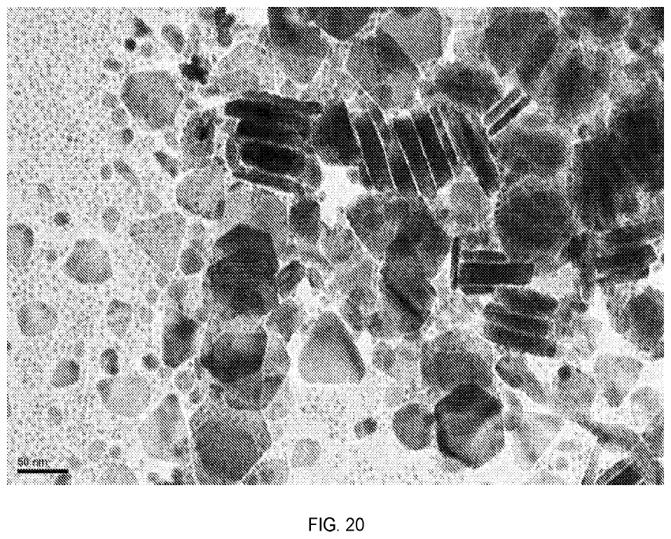

FIG. 20 shows the product from the synthesis of CdSe NCs with ligand 20e.

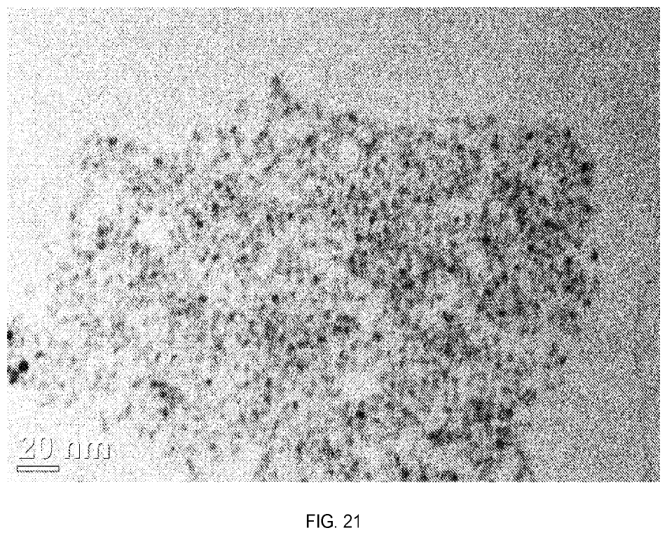

FIG. 21 shows 20h-capped 2.5 nm CdSe NCs drop-cast from a dilute dispersion.

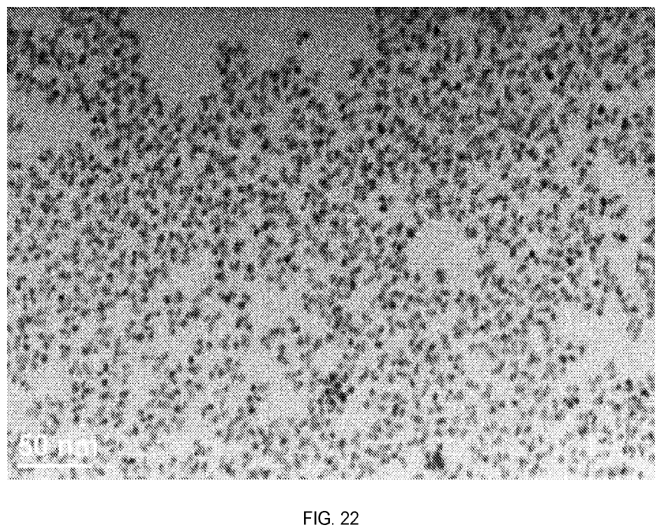

FIG. 22 shows 20j-capped 2.8 nm CdSe NCs drop-cast from a dilute dispersion.

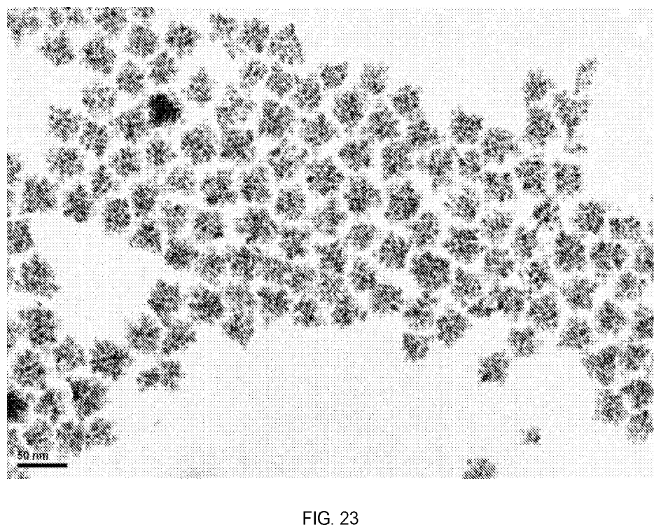

FIG. 23 shows the product from the synthesis of CdSe NCs with ligand 20k.

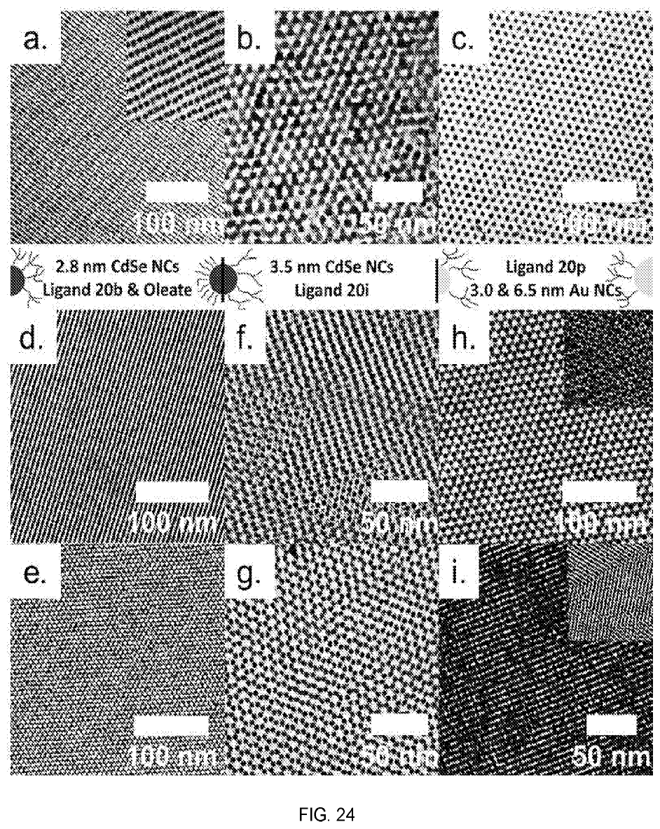

FIG. 24 shows (a) (001) projection of bcc 20b-capped 2.8 nm CdSe, with inset of 20b-capped 2.4 nm CdS, (b) (001) projection of hcp 20i-capped 7.0 nm ZnO, and (c) close-packed hexagonal monolayer of 20b-capped 5.5 nm PbSe NCs. (d) bcc-type self-assembly of 2.8 nm CdSe NCs prepared by direct synthesis with ligand 20a. (e) hcp-type assembly of 2.8 nm CdSe NCs prepared by synthesis with oleic acid ligands. (f, g) 3.5 nm CdSe NCs capped with 20i ligand self-assembled into hcp, fcc, and bcc superlattices. (h) hcp superlattice of 20o-capped 6.5 nm Au NCs. Inset shows an fcc assembly of the same sample. (i) 20o-capped 3.0 nm Au NCs in a bcc superlattice. The inset shows another region of bcc superlattices of the sample including a characteristic twin boundary.

### Example 12. Formation of Binary Nanocrystal Superlattices (BNSLs)

The BNSLs of the present example were formed using the same procedures, i.e. casting on to a DEG surface or by dropcasting dilute dispersions of NCs directly on to carbon-coated Cu TEM grids, described in Example 11, except that two types of nanocrystals were prepared in hexanes at a defined stoichiometry and concentration (typically 5-10 mg/mL). The two types of NCs used to form the BNSL may both be inventive NCs made according to Examples 3-9. The two types of NCs may also differ only in size. Alternatively, one type of NC may be an inventive NC made according to Examples 3-9 and the other type may be an NC prepared using a commercially-available ligand.

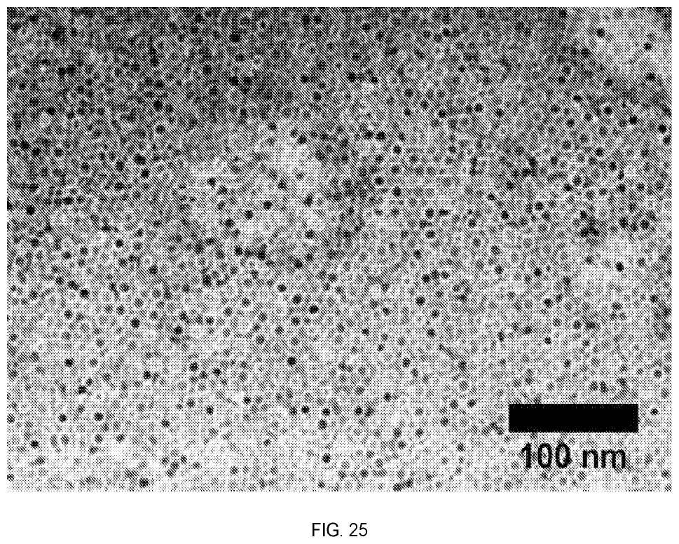

FIG. 25 shows the spontaneous formation of CaCu₅-like BNSL domains in a drop-casted sample of CdSe NCs synthesized with ligand 20l. The particle size distribution formed in the synthesis is bimodal.

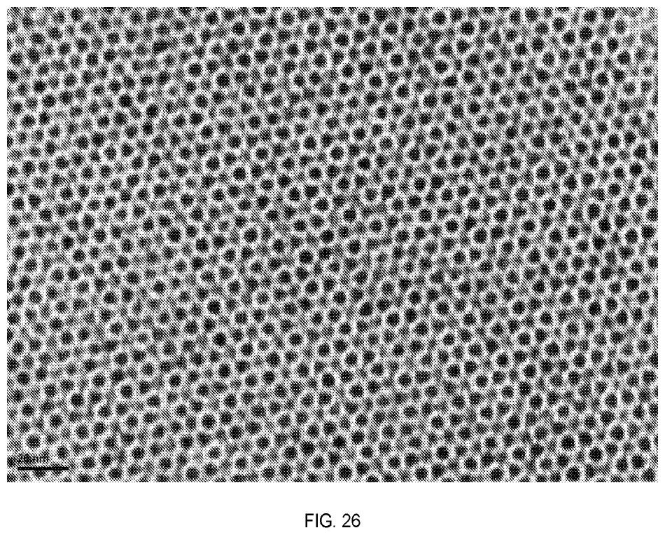

FIG. 26 shows MgZn₂-type BNSL self-assembled from oleate-capped 5.3 nm CdSe NCs and 20d-capped 2.4 nm CdSe NCs.

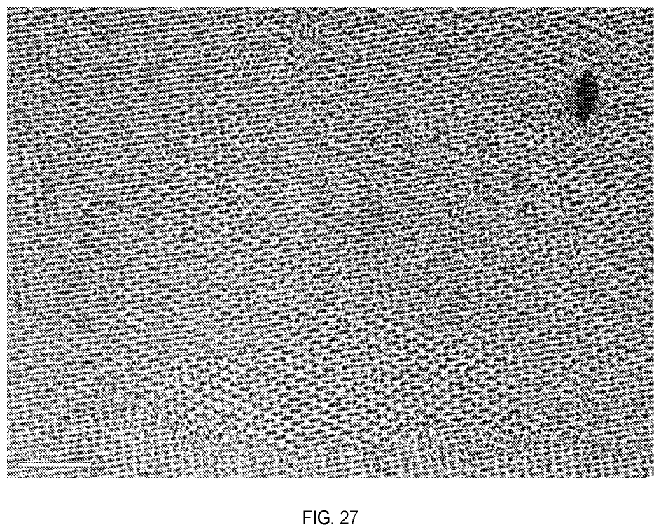

FIG. 27 shows MgZn₂-type BNSLs self-assembled from 20b-capped 3.5 nm PbSe NCs and 20b-capped 2.8 nm CdSe NCs.

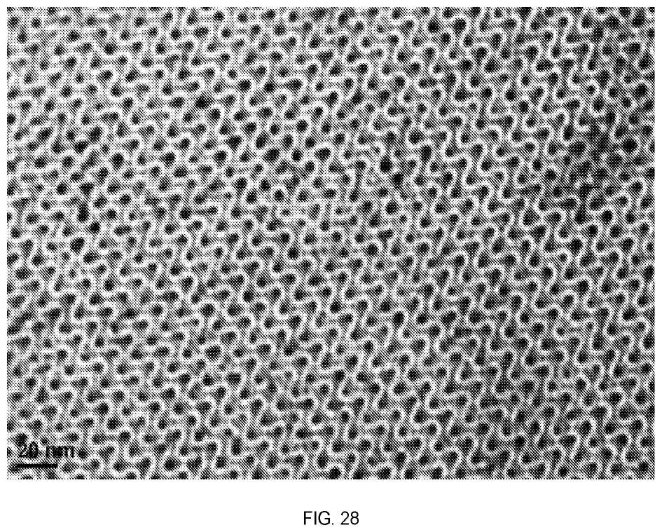

FIG. 28 shows a possible AlB₂-type BNSL self-assembled from 20b-capped 5.5 nm PbSe NCs and 20d-capped 2.4 nm CdSe NCs.

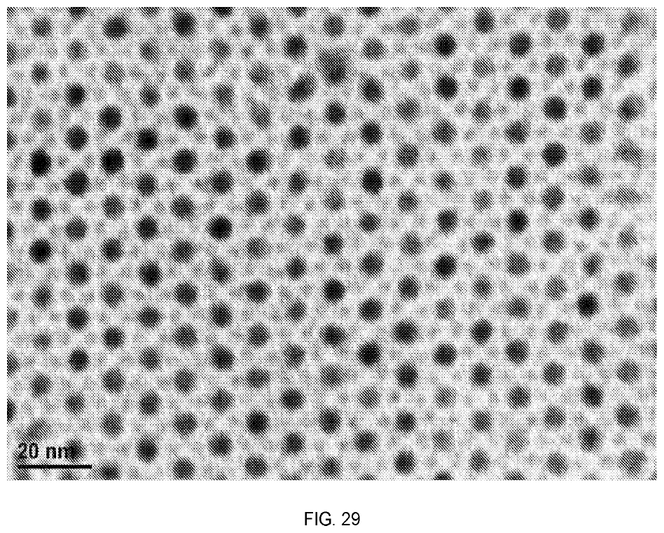

FIG. 29 shows an AlB₂-type BNSL self-assembled from 20b-capped 5.5 nm PbSe NCs and 20d-capped 2.4 nm CdSe NCs.

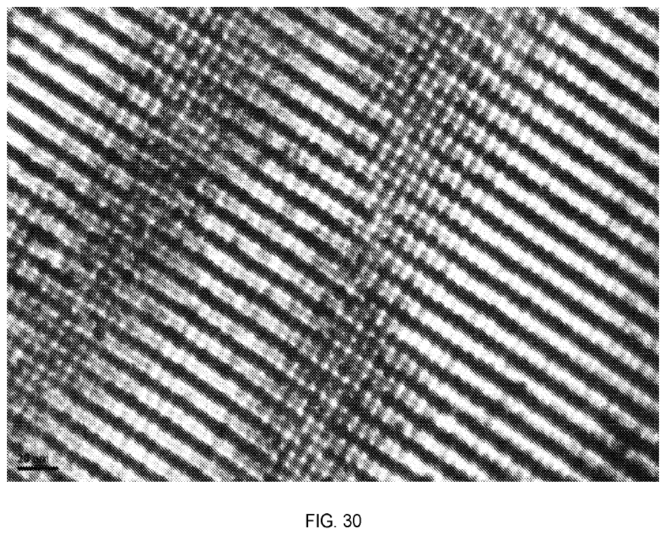

FIG. 30 shows CuAu BNSLs with antiphase boundaries formed from 20o-capped 6.5 nm Au NCs and oleate-capped 2.8 nm CdSe NCs.

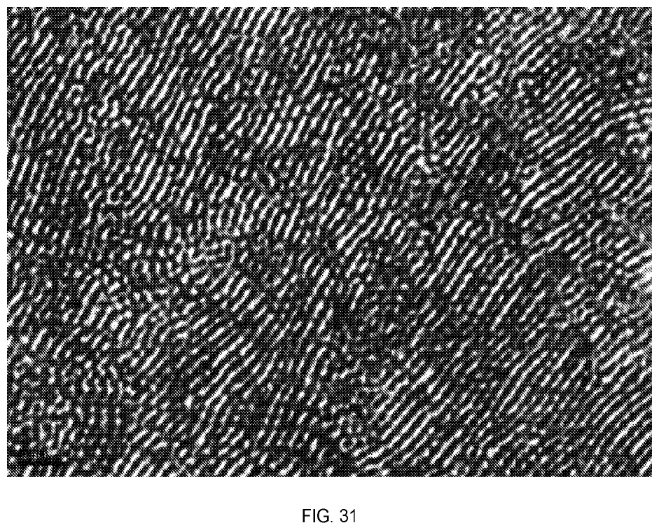

FIG. 31 shows a binary liquid crystalline phase formed from 20o-capped 6.5 nm Au NCs and oleate-capped 2.8 nm CdSe NCs. Approximate composition is 1:1.

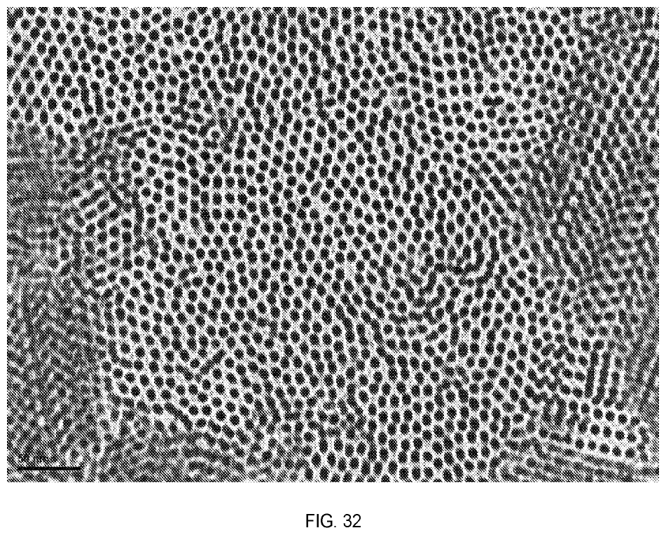

FIG. 32 shows a binary liquid crystalline phase formed from 20o-capped 6.5 nm Au NCs and oleate-capped 2.8 nm CdSe NCs. Approximate composition is 1 Au: 2 CdSe.

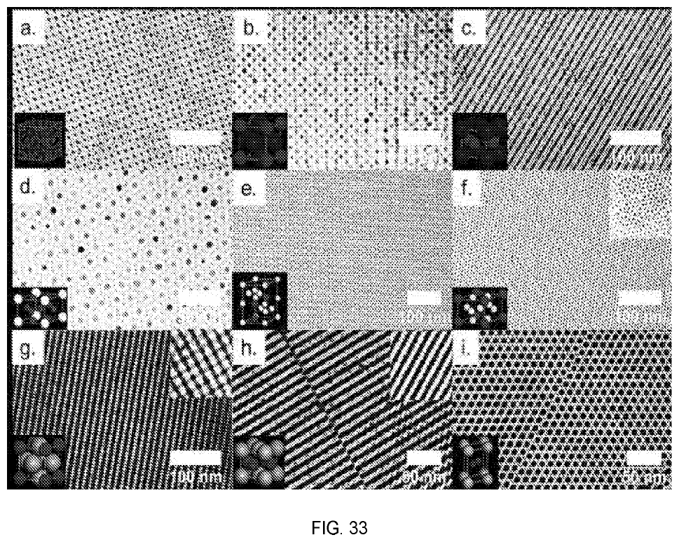

FIG. 33 shows TEM micrographs of (a) NaZn₁₃, (b) Cu₃Au, and (c) CuAu BNSLs composed of different stoichiometries of 20b-capped 5.5 nm PbSe and 20d-capped 2.4 nm CdSe NCs; (d) CaCu₅ BNSL composed of 20i-capped 3.5 nm CdSe and 20i-capped 7.0 nm ZnO NCs; (e) MgZn₂ and (f) CaCu₅ BNSLs composed of different stoichiometries of 20i-capped 3.5 nm CdSe and 20b-capped 2.4 nm CdS NCs. The inset of (f) shows 12-fold symmetric defects which occur in mixtures of the two NCs. (g) Cu₃Au-type, (h) CuAu-type, and (i) AlB₂-type BNSLs formed from different stoichiometries of oleate-capped 2.8 nm CdSe and 20o-capped 6.5 nm Au NCs. Insets of (g) and (h) show images from other BNSL domains of the same type. Cartoons of the unit cell for each crystal structure are inset at the bottom left of the images.

The data for the BNSLs formed according to the present example are summarized in Table 2.

TABLE 2

| Large | Small | | | | | | | |
|---|---|---|---|---|---|---|---|---|
| NCs | NCs | d_{large} | dₛₘₐₗₗ | t_{large} | tₛₘₐₗₗ | Y^{a} | S_{AB} | BNSL(s) |
| oleate- | 20d- | 5.3 | 2.4 | 1.0 | 1.4 | 0.71 | 1.4 | MgZn₂ |
| CdSe | CdSe | | | | | | | |
| 20o-Au | 20d- | 6.5 | 2.4 | 2.2 | 1.4 | 0.48 | 0.64 | CuAu, |
| | CdSe | | | | | | | Cu₃Au |
| 20o-Au | oleate- | 6.5 | 2.8 | 2.2 | 1.0 | 0.44 | 0.45 | CuAu, AlB₂, |
| | CdSe | | | | | | | Cu₃Au |
| 20b- | 20d- | 5.5 | 2.4 | 2.0 | 1.4 | 0.55 | 0.7 | NaZn₁₃, |
| PbSe | CdSe | | | | | | | Cu₃Au, AlB₂, |
| | | | | | | | | CuAu |
| 20i- | 20b- | 3.5 | 2.4 | 1.85 | 2.0 | 0.89 | 1.08 | CaCu₅, |
| CdSe | CdS | | | | | | | MgZn₂ |
| 20i- | 20i- | 7 | 3.5 | 1.85 | 1.85 | 0.67 | 1 | CaCu₅ |
| ZnO | CdSe | | | | | | | |
| 20b- | 20b- | 3.5 | 2.7 | 2.0 | 2.0 | 0.89 | 1 | MgZn₂ |
| PbSe | CdSe | | | | | | | |
| 20o-Au | 20b- | 6 | 5.4 | 2.2 | 2.0 | 0.90 | 0.91 | CuAu |
| | CdSe | | | | | | | |

^{a}combined diameter of the inorganic core and thicknesses of the shell of the small NC divided by the combined diameter of the large NC.

### Example 13. Use of Microreactors for Synthesizing Rare Earth Nanoparticles

To test the feasibility of the microreaction setup in producing monodisperse rare earth-containing nanoparticles, dysprosium fluoride (DsF₃) rare-earth nanoplates were chosen as the model system. Their high sensitivity to reaction conditions, such as volume, indeed makes them an excellent candidate for investigating the feasibility of the microreactors for screening rare earth nanoparticle morphologies.

DyF₃ nanocrystals were synthesized through thermal decomposition of dysprosium trifluoroacetate precursors in the presence of lithium fluoride and oleic acid in a traditional solvothermal synthesis reaction vessel and in a microreactor vessel to determine the effect of volume on the morphology of the formed nanocrystals.

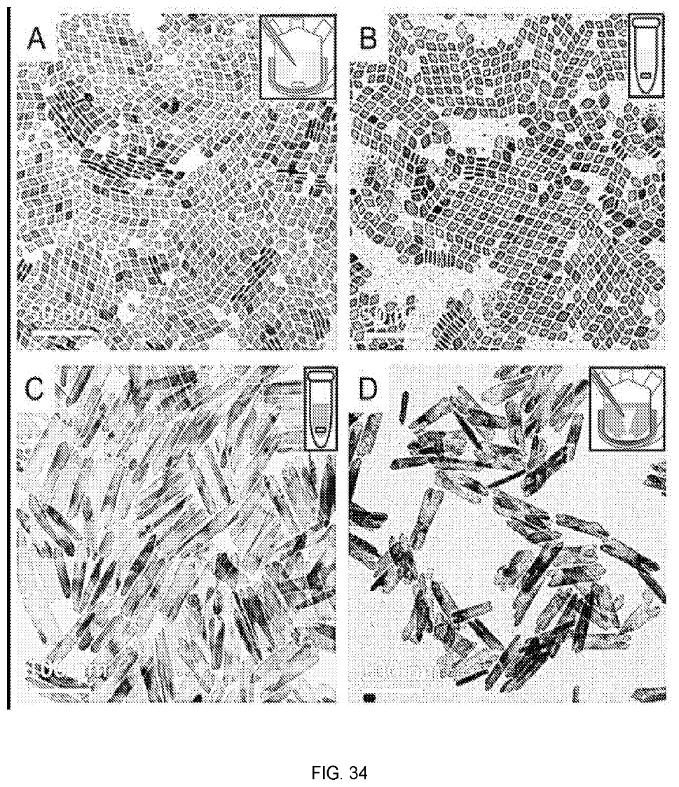

The size, shape, and monodispersity of the nanocrystals formed in the traditional solvothermal synthesis reaction vessel and of those formed in the microreactor vessel were very similar. The DyF₃ nanocrystals formed in the traditional synthesis vessel exhibited the expected rhombic plate morphology and are shown in FIG. 34A. The DyF₃ nanocrystals formed in the microreactor, which was subjected to the same temperature (310° C.) for the same amount of time (40 min) as in the traditional synthesis approach, are shown in FIG. 34B.

In another instance, DyF₃ nanocrystals were made in both the traditional solvothermal synthesis reaction vessel and the microreactor. The oleic acid was replaced by a molar equivalent of inventive compound 20b such that the molar ratio of compound 20b to oleic acid was 50:50. Again, the size, shape, and monodispersity of the nanocrystals formed in the traditional solvothermal synthesis reaction vessel and of those formed in the microreactor vessel were similar. In both cases, the DyF₃ particles were in the form of elongated plates. The DyF₃ elongated plates obtained from an equimolar ratio of oleic acid and compound 20b in a microreactor and in the traditional solvothermal reaction vessel are shown in FIGS. 34C and 34D, respectively.

This result shows that rare earth nanocrystals can be formed in microreactors with minimal change to their morphology.

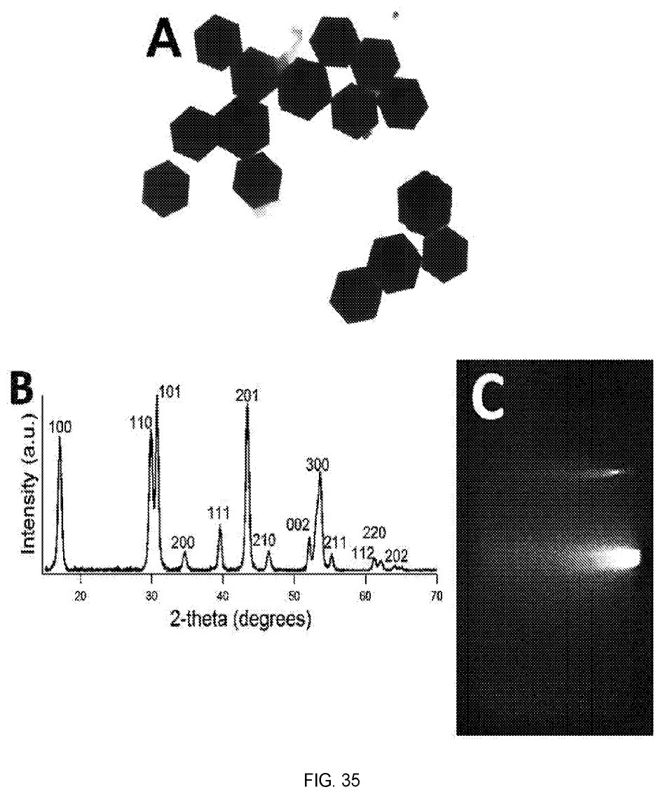

The microreactor setup described herein was also found to be suitable for the synthesis of β-NaYF₄. High temperatures are of interest for synthesizing doped upconverting β-NaYF₄ as a high temperature ramp rate is required to co-nucleate the rare-earth elements. The β-NaYF4 nanoparticles were prepared at a temperature of 340° C. and the reaction time was 1 h. The microreactor vessels are capable of producing β-NaYF₄ nanocrystals through solvothermal synthesis, as shown in FIG. 35.

### Example 14. Effect of Inventive Compound 20b on Rare Earth Nanocrystal Morphology

The effect of inventive compound 20b on the morphology of formed nanocrystals was investigated.

Initially, the desired rare earth trifluoroacetates were made. Approximately ten grams of rare earth oxide was added to 100mL of a 1:1 solution of distilled water and trifluoroacetic acid (TFA) in a round bottom flask. This suspension was heated to 80° C. and stirred until clear. Once the solution became clear, the solution was allowed to cool to room temperature, and the solvent was then evaporated off, leaving the solid rare earth trifluoroacetate behind.

Generally, the rare earth trifluoroacetate (36 μmol) and LiF (38 μmol) were added into a borosilicate test tube with a micro stir bar and 300 μL of a 1:1 oleic acid (OA)/1-octadecene (ODE) mixture. A series of microreactors were prepared in which a molar equivalent of oleic acid was replaced by compound 20b with the amount of replacement being varied across the series. The test tubes were connected to a Schlenk line and placed under vacuum while in a 100° C. silicone oil bath for 45 min. The microreactors were then filled with N₂ gas and placed in a 310° C. 1:1 KNO₃:NaNO₃ salt bath for 40 min. The reactions were quenched by adding 1 mL of ODE. The nanoparticles were isolated from each reaction mixture by adding 40mL of a 1:1 hexanes and ethanol solution and centrifuging at 6000 rpm for 2 min. The resulting nanoparticles were dispersed in hexanes and no further size-selective precipitation strategies were employed.

In one instance, the effect of inventive compound 20b on the formation of DyF₃ nanocrystals was investigated.

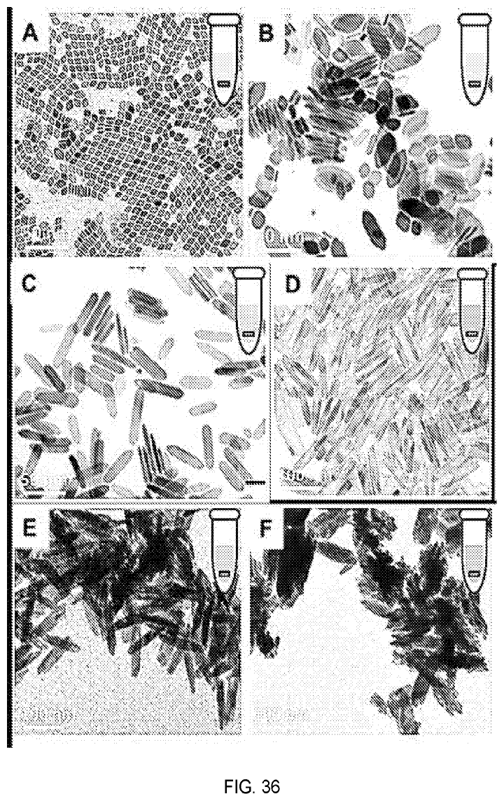

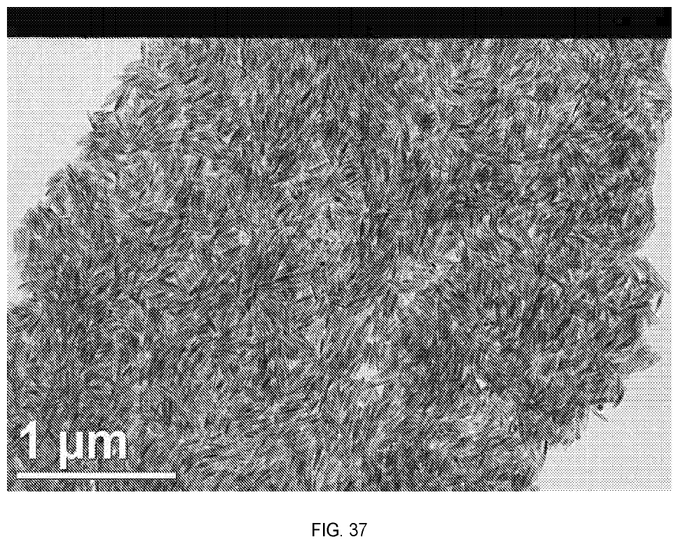

It was surprisingly found that as the proportion of compound 20b replacing the oleic acid increased, the morphology of the resulting DyF₃ became more rod-like, eventually leading to large aggregates of fused particles at 100% of ligand replacement, as shown in FIG. 36. Of particular note are the resulting particles from the equimolar mixture (50:50) of oleic acid and compound 20b in the reaction solution (see FIGS. 34C and 34D). The resulting elongated plates can be obtained in high yield and with low-size dispersity, as indicated by the low-magnification transmission electron microscopy (TEM) image (see FIG. 37). The powder diffraction pattern of these samples, shown in FIG. 38, confirms that the DyF₃ elongated nanoplates maintain the same a-phase crystallinity as the rhombic platelets.

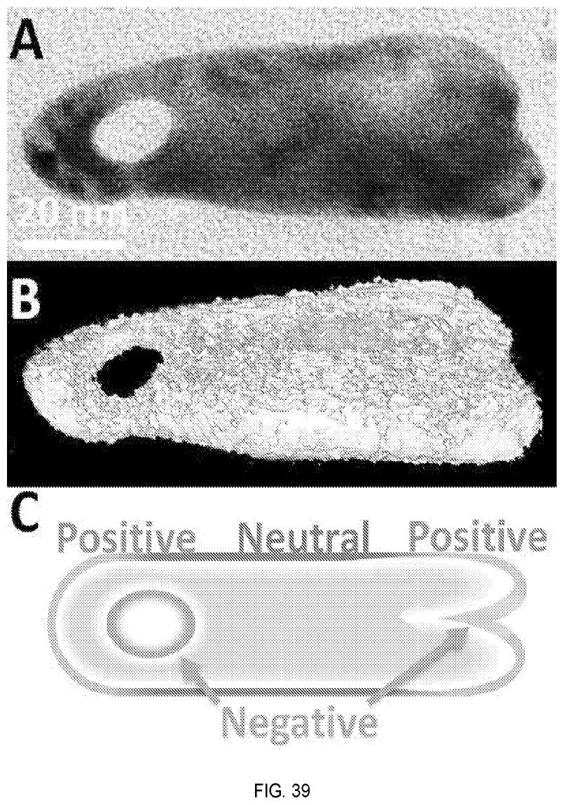

A closer look at the DyF₃ elongated plates obtained by using an equimolar mixture of oleic acid and inventive compound 20b reveals their distinguishing characteristics, such as concave curvature features and incomplete growth. FIG. 39 highlights a DyF₃ elongated plate that displays both occurrences. FIG. 39A shows the presence of both a hole in the body of the plate and termination with a concave curvature. These features are present at a large scale, as seen in FIG. 37, but the degree of curvature varies between plates. Tilt tomography highlights these features, as shown in the 3D reconstruction shown in FIG. 39B. The schematic shown in FIG. 39C highlights the apparent positive, negative, and neutral curvatures of the edges of the elongated plate, which may have important implications about the surface chemistry of these plates. For example, Rotz et al. (Rotz, M. W.; Culver, K. S. B.; Parigi, G.; MacRenaris, K. W.; Luchinat, C.; Odom, T. W.; Meade, T. J. ACS Nano 2015, 9 (3), 3385-3396) have reported that the negative curvature of gold nanostars coated in Gd (III) labelled DNA limits water sequestration during magnetic measurements. The negative curvature present in the DyF₃ elongated plates may offer similar protection.

### Example 15. Synthesis of Various Rare Earth Nanocrystals

Various rare earth nanocrystals were synthesized according to the general method used in Example 14, except that the molar ratio of compound 20b to oleic acid was fixed at 50:50.

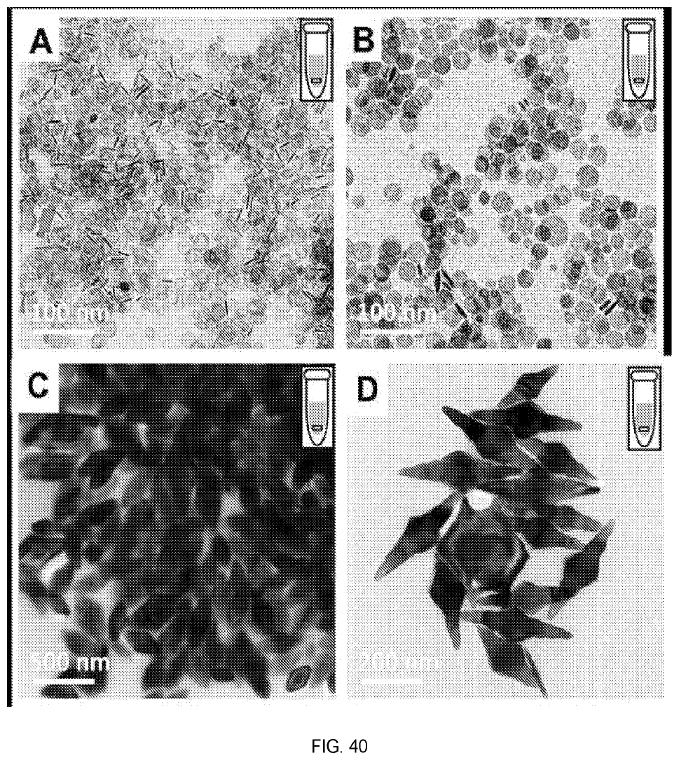

It was discovered that well-defined monodisperse rare earth nanocrystals can be formed from La, Eu, Y, and Er. Circular plates of LaF₃ and EuF₃ and octahedral LiYF₄ and LiErF₄ are shown in FIG. 40A-40D, respectively.

### Example 16. Effect of Reaction Time on Morphology Rare Earth Nanocrystals

In accordance with the present invention, new morphologies for rare earth nanocrystals may be possible.

Rare earth nanocrystals were synthesized according to the general method used in Example 14, except that the molar ratio of compound 20b to oleic acid was fixed at 50:50 and the reaction time was 10 minutes.

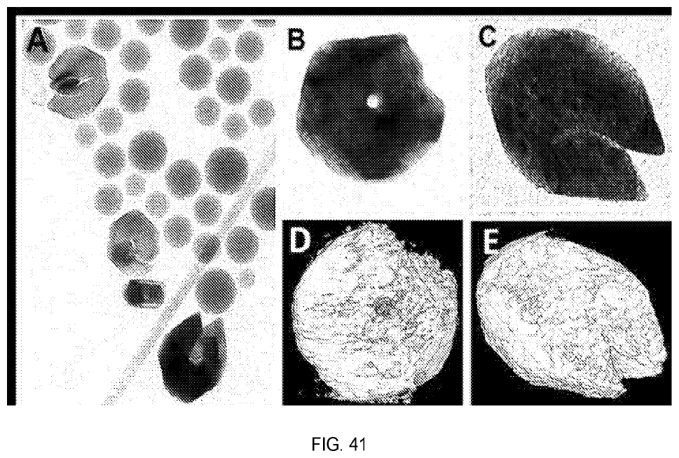

The formed particles shown in FIG. 41 present evidence of a distinct growth pathway, such as screw dislocation, fused growth, or epitaxy, that was induced by the presence of compound 20b in the mixture. Its presence at shorter reaction times may explain the defects present in the elongated plates. The tilt series and 3D reconstructions presented in FIGS. 41D and 41E indicate that these distinct nanoparticles have a mismatch between the fused edges, adding support for the screw-dislocation growth mechanism.

As shown herein, microreactors allow for the investigation of solvothermal reaction conditions via a tandem parallel heating source. Monodisperse anisotropic rare earth nanocrystals can be obtained from this method, and these results scale up effectively. The microreactor enables more advanced and controlled studies of the reaction parameters that direct high-temperature growth of nanocrystals. Also shown herein are inventive polycatenar compounds that result in distinct morphologies of DyF₃ nanocrystals, such as elongated plates and screw-dislocated nanoparticles. The pilot reaction conditions can be extended to other rare earth elements to access other well-controlled shapes such as octahedral, circular plates, and square bipyramidal nanocrystals.

1. A compound represented by formula (I) 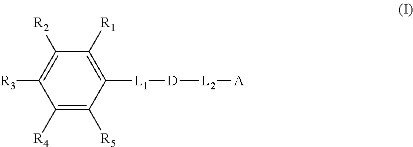
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
D is a divalent moiety selected from the group consisting of 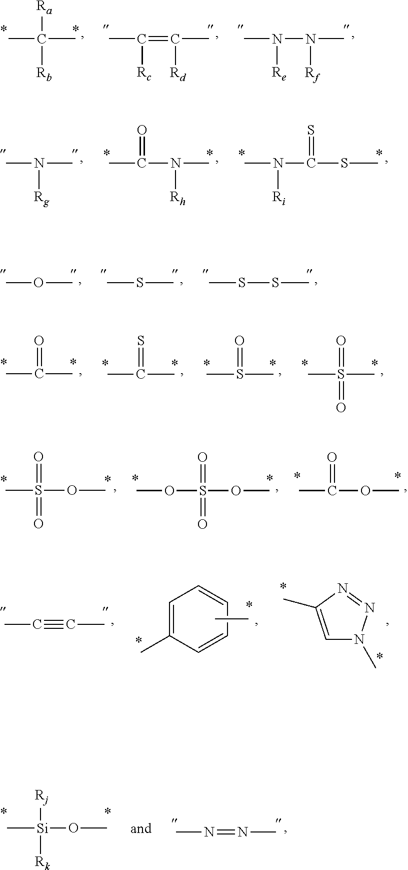
wherein each occurrence of Rₐ-Rₖ are each, independently, H, halogen, or hydrocarbyl; and
A is —COOR₇, —NR₈R₉, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl.

2. The compound according to claim 1, wherein R₁, R₂, R₃, R₄, and R₅ are each, independently, H, alkyl, fluoroalkyl, or —OR₆, wherein each occurrence of R₆ is alkyl, arylalkyl, or fluoroalkyl.

3. (canceled)

4. The compound according to claim 1, wherein L₁ and L₂ are each, independently, a bond or (C₁-C₁₀)alkylene.

5. The compound according to claim 1, wherein D is

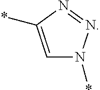 [SMILES: *c1cn(*)nn1]

6. The compound according to claim 1, wherein A is —COOR₇, —NR₈R₉, —CN, —SR₁₂ or —OR₁₆, wherein each occurrence of R₇, R₈, R₉, R₁₀, and R₁₆ is aryl.

17. A method for producing the compound according to claim 1, the method comprising:
reacting a compound represented by the structure of formula (II): 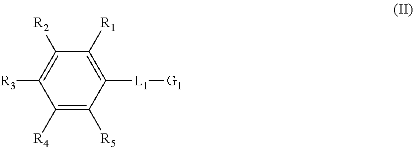
with a compound represented by the structure of formula (III):
G₂-L₂-A (III)
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
A is —COOR₇, —NR₈R₉, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl; and
each occurrence of G₁ is a reactive group capable of reacting with the reactive group G₂, and
G₂ is a reactive group capable of reacting with the reactive group G₁.

8. The method according to claim 7, wherein G₁ is a reactive group selected from the group consisting of —X, —NH₂, —N₃, —(C═O)X, -Ph(C═O)X, —SH, —CH═CH₂, and —C≡CH; wherein X is a leaving group.

9. The method accordinvg to claim 7, wherein G₂ is a reactive group selected from the group consisting of —(C═O)X, —CH═CH₂, —C≡CH, —NH₂, —N₃, -Ph(C═O)X, —Sh, —X, —NCO, —NCS; wherein X is a leaving group.

10. A hybrid nanoparticle comprising:
(a) a metallic core, and
(b) a compound according to claim 1, attached to the surface of the metallic core.

11. The hybrid nanoparticle according to claim 10, wherein the metallic core comprises a transition metal chalcogenide.

12. The hybrid nanoparticle according to claim 10, wherein the metallic core is a nanocrystal.

13. A method for producing the hybrid nanoparticle according to claim 10, the method comprising:
forming the metallic core in the presence of a compound represented by formula (I) 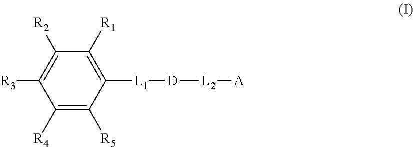
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
D is a divalent moiety selected from the group consisting of 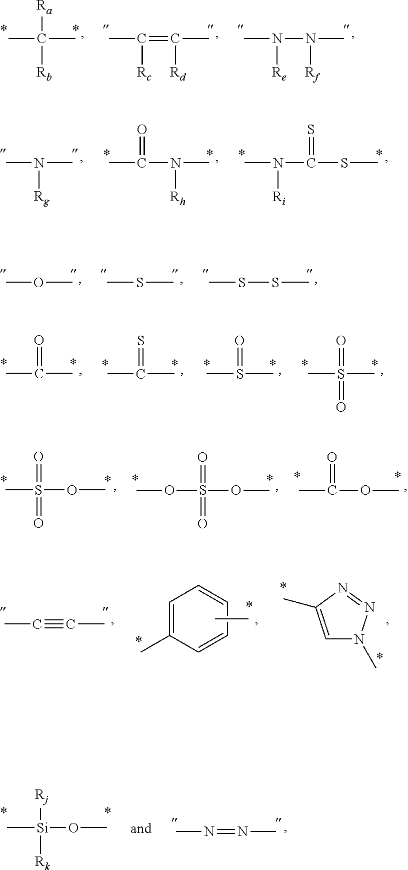
wherein each occurrence of Rₐ-Rₖ are each, independently, H, halogen, or hydrocarbyl; and
A is —COOR₇, —NR₈R₉, —PO₃R₁₀R₁₁, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl; thereby producing the hybrid nanoparticle.

14. The method according to claim 13, wherein the method does not comprise any ligand exchange step.

15. A film comprising a plurality of hybrid nanoparticles according to claim 10.

16.-18. (canceled)

19. A method for making nanoparticles comprising a rare earth element, the method comprising:
(a) heating one or more reaction vessels, each said vessel containing a reaction mixture comprising a rare earth-containing precursor compound, and
(b) recovering the nanoparticles formed in the one or more reaction vessels in step (a).

20. The method according to claim 19, wherein the heating source is a salt bath.

21. The method according to claim 19, wherein each reaction mixture further comprises a compound represented by formula (I) 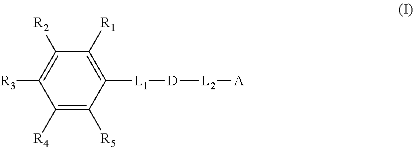
wherein
R₁, R₂, R₃, R₄, and R₅ are each, independently, H, hydrocarbyl, halogenated hydrocarbyl, or —OR₆, wherein each occurrence of R₆ is hydrocarbyl or halogenated hydrocarbyl;
L₁ and L₂ are each, independently, a bond or hydrocarbylene;
D is a divalent moiety selected from the group consisting of 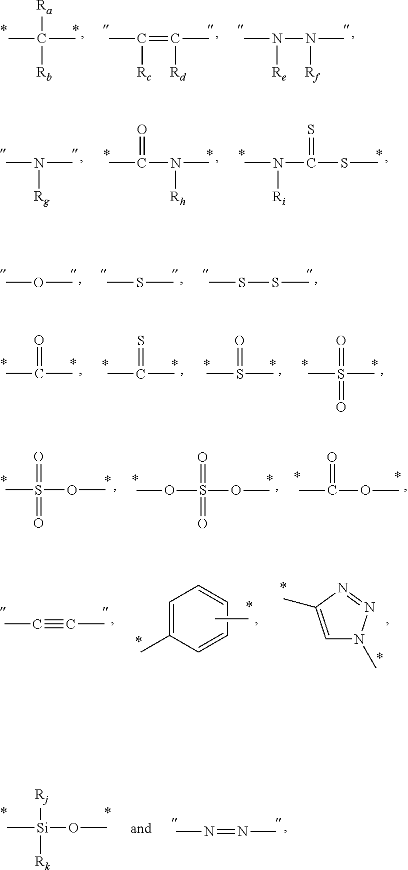
wherein each occurrence of Rₐ-Rₖ are each, independently, H, halogen, or hydrocarbyl; and
A is —COOR₇, —NR₈R₉, —PO₃R₁₀R₁₁, —CN, —SR₁₂, —C(SR₁₃)CH₂(SR₁₄), —Si(OR₁₅)₃, —H or —OR₁₆, wherein each occurrence of R₇-R₁₆ are each, independently, H or hydrocarbyl.

22. The method according to claim 19, wherein each reaction mixture further comprises oleic acid.

23. The method according to claim 22, wherein the molar ratio of the compound represented by formula (I), when present, relative to oleic acid, when present, is from 99:1 to 20:80.

24. Nanoparticles obtained by the method according to claim 19.
